(19) United States
(12) Patent Application Publication
(10) Pub. No.: US 2002/0150944 A1
(43) Pub. Date: Oct. 17, 2002

(54) Biochemical analysis unit and method for exposing stimulable phosphor sheet using the same

(75) Inventor: Yuichi Hosoi, Kanagawa (JP)

Correspondence Address: SUGHRUE MION, PLLC, 2100 PENNSYLVANIA AVENUE, N.W., WASHINGTON, DC, 20037, US

(73) Assignee: FUJI PHOTO FILM CO., LTD.

(21) Appl. No.: 10/115,964

(22) Filed: Apr. 5, 2002

(30) Foreign Application Priority Data
Apr. 6, 2001 (JP) 2001-108974

(51) Int. Cl. C12Q 1/68; C12M 1/34

(52) U.S. Cl. 435/6; 435/287.2

(57) ABSTRACT

A biochemical analysis unit includes a plurality of absorptive regions formed of absorptive material and spaced apart from each other and a plurality of isolating regions formed of a material capable of attenuating radiation energy and/or light energy for isolating the plurality of absorptive regions, the plurality of isolating regions being formed so that surfaces thereof lie outward of surfaces of the individual absorptive regions. According to the thus constituted biochemical analysis unit, it is possible to effectively prevent electron beams released from a radioactive labeling substance or chemiluminescent emission released from the plurality of absorptive regions from being scattered and to produce biochemical analysis data free from noise by scanning a stimulable phosphor layer exposed to electron beams or chemiluminescent emission released from the plurality of absorptive regions with a stimulating ray and photoelectrically detecting stimulated emission released from the stimulable phosphor layer.

### BACKGROUND OF THE INVENTION

The present invention relates to a biochemical analysis unit and a method for exposing a stimulable phosphor sheet using the same and, particularly, to a biochemical analysis unit and a method for exposing a stimulable phosphor sheet using the same which can prevent noise caused by the scattering of electron beams released from a radioactive labeling substance from being generated in biochemical analysis data even in the case of forming a plurality of spot-like regions containing specific binding substances on the surface of a carrier at a high density, which can specifically bind with a substance derived from a living organism and whose sequence, base length, composition and the like are known, specifically binding the specific binding substances contained in the plurality of spot-like regions with a substance derived from a living organism labeled with a radioactive substance to selectively label the spot-like specific binding substances with the radioactive substance, thereby obtaining a biochemical analysis unit, superposing the thus obtained biochemical analysis unit and a stimulable phosphor layer, exposing the stimulable phosphor layer to the radioactive labeling substance, irradiating the stimulable phosphor layer with a stimulating ray to excite the stimulable phosphor, photoelectrically detecting the stimulated emission released from the stimulable phosphor layer to produce biochemical analysis data, and analyzing the substance derived from a living organism, and a biochemical analysis unit and a method for exposing a stimulable phosphor sheet using the same which can prevent noise caused by the scattering of chemiluminescent emission released from a plurality of spot-like regions of a biochemical analysis unit from being generated in biochemical analysis data even in the case of forming the plurality of spot-like regions containing specific binding substances on the surface of a carrier at a high density, which can specifically bind with a substance derived from a living organism and whose sequence, base length, composition and the like are known, specifically binding the specific binding substances contained in the plurality of spot-like regions with a substance derived from a living organism labeled with a labeling substance which generates chemiluminescent emission when it contacts a chemiluminescent substrate to produce a biochemical analysis unit, causing a chemiluminescent substrate to come into contact with the biochemical analysis unit, thereby causing the plurality of spot-like regions of the biochemical analysis unit to release chemiluminescent emission, holding the biochemical analysis unit whose plurality of spot-like regions are releasing chemiluminescent emission in close contact with a stimulable phosphor layer, exposing the stimulable phosphor layer to chemiluminescent emission, irradiating the stimulable phosphor layer with a stimulating ray, photoelectrically detecting stimulated emission released from the stimulable phosphor layer to produce biochemical analysis data, and analyzing the substance derived from a living organism.

### DESCRIPTION OF THE PRIOR ART

An autoradiographic analyzing system using as a detecting material for detecting radiation a stimulable phosphor which can absorb, store and record the energy of radiation when it is irradiated with radiation and which, when it is then stimulated by an electromagnetic wave having a specified wavelength, can release stimulated emission whose light amount corresponds to the amount of radiation with which it was irradiated is known, which comprises the steps of introducing a radioactively labeled substance into an organism, using the organism or a part of the tissue of the organism as a specimen, superposing the specimen and a stimulable phosphor sheet formed with a stimulable phosphor layer for a certain period of time, storing and recording radiation energy in a stimulable phosphor contained in the stimulable phosphor layer, scanning the stimulable phosphor layer with an electromagnetic wave to excite the stimulable phosphor, photoelectrically detecting the stimulated emission released from the stimulable phosphor to produce digital image signals, effecting image processing on the obtained digital image signals, and reproducing an image on displaying means such as a CRT or the like or a photographic film (see, for example, Japanese Patent Publication No. 1-60784, Japanese Patent Publication No. 1-60782, Japanese Patent Publication No. 4-3952 and the like).

There is further known chemiluminescence analysis system comprising the steps of employing, as a detecting material for light, a stimulable phosphor which can absorb and store the energy of light upon being irradiated therewith and release a stimulated emission whose amount is proportional to that of the received light upon being stimulated with an electromagnetic wave having a specific wavelength range, selectively labeling a fixed high molecular substance such as a protein or a nucleic acid sequence with a labeling substance which generates chemiluminescent emission when it contacts a chemiluminescent substance, contacting the high molecular substance selectively labeled with the labeling substance and the chemiluminescent substance, storing and recording the chemiluminescent emission in the wavelength of visible light generated by the contact of the chemiluminescent substance and the labeling substance in the stimulable phosphor contained in a stimulable phosphor layer formed on a stimulable phosphor sheet, scanning the stimulable phosphor layer with an electromagnetic wave to excite the stimulable phosphor, photoelectrically detecting the stimulated emission released from the stimulable phosphor to produce digital signals, effecting data processing on the obtained digital signals, and reproducing data on displaying means such as a CRT or a recording material such as a photographic film (see for example, U.S. Pat. No. 5,028,793, UK Patant Application 2,246,197 A and the like).

Unlike the system using a photographic film, according to these systems using the stimulable phosphor as a detecting material, development, which is chemical processing, becomes unnecessary. Further, it is possible reproduce a desired image by effecting image processing on the obtained image data and effect quantitative analysis using a computer. Use of a stimulable phosphor in these processes is therefore advantageous.

On the other hand, a fluorescence analyzing system using a fluorescent substance as a labeling substance instead of a radioactive labeling substance in the autoradiographic analyzing system is known. According to this system, it is possible to study a genetic sequence, study the expression level of a gene, and to effect separation or identification of protein or estimation of the molecular weight or properties of protein or the like. For example, this system can perform a process including the steps of distributing a plurality of DNA fragments on a gel support by means of electrophoresis after a fluorescent dye was added to a solution containing a plurality of DNA fragments to be distributed, or distributing a plurality of DNA fragments on a gel support containing a fluorescent dye, or dipping a gel support on which a plurality of DNA fragments have been distributed by means of electrophoresis in a solution containing a fluorescent dye, thereby labeling the electrophoresed DNA fragments, exciting the fluorescent dye by a stimulating ray to cause it to release fluorescent light, detecting the released fluorescent light to produce an image and detecting the distribution of the DNA fragments on the gel support. This system can also perform a process including the steps of distributing a plurality of DNA fragments on a gel support by means of electrophoresis, denaturing the DNA fragments, transferring at least a part of the denatured DNA fragments onto a transfer support such as a nitrocellulose support by the Southern-blotting method, hybridizing a probe prepared by labeling target DNA and DNA or RNA complementary thereto with the denatured DNA fragments, thereby selectively labeling only the DNA fragments complementary to the probe DNA or probe RNA, exciting the fluorescent dye by a stimulating ray to cause it to release fluorescent light, detecting the released fluorescent light to produce an image and detecting the distribution of the target DNA on the transfer support. This system can further perform a process including the steps of preparing a DNA probe complementary to DNA containing a target gene labeled by a labeling substance, hybridizing it with DNA on a transfer support, combining an enzyme with the complementary DNA labeled by a labeling substance, causing the enzyme to contact a fluorescent substance, transforming the fluorescent substance to a fluorescent substance having fluorescent light releasing property, exciting the thus produced fluorescent substance by a stimulating ray to release fluorescent light, detecting the fluorescent light to produce an image and detecting the distribution of the target DNA on the transfer support. This fluorescence detecting system is advantageous in that a genetic sequence or the like can be easily detected without using a radioactive substance.

Similarly, there is known a chemiluminescence detecting system comprising the steps of fixing a substance derived from a living organism such as a protein or a nucleic acid sequence on a support, selectively labeling the substance derived from a living organism with a labeling substance which generates chemiluminescent emission when it contacts a chemiluminescent substrate, contacting the substance derived from a living organism and selectively labeled with the labeling substance and the chemiluminescent substrate, photoelectrically detecting the chemiluminescent emission in the wavelength of visible light generated by the contact of the chemiluminescent substrate and the labeling substance to produce digital image signals, effecting image processing thereon, and reproducing a chemiluminescent image on a display means such as a CRT or a recording material such as a photographic film, thereby obtaining information relating to the high molecular substance such as genetic information

Further, a micro-array analyzing system has been recently developed, which comprises the steps of using a spotting device to drop at different positions on the surface of a carrier such as a slide glass plate, a membrane filter or the like specific binding substances, which can specifically bind with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, thereby forming a number of independent spots, specifically binding the specific binding substances using a hybridization method or the like with a substance derived from a living organism such as a cell, virus, hormone, tumor marker, enzyme, antibody, antigen, abzyme, other protein, a nuclear acid, cDNA, DNA or mRNA, which is gathered from a living organism by extraction, isolation or the like or is further subjected to chemical processing, chemical modification or the like and which is labeled with a labeling substance such as a fluorescent substance, dye or the like, thereby forming a micro-array, irradiating the micro-array with a stimulating ray, photoelectrically detecting light such as fluorescence emission released from a labeling substance such as a fluorescent substance, dye or the like, and analyzing the substance derived from a living organism. This micro-array analyzing system is advantageous in that a substance derived from a living organism can be analyzed in a short time period by forming a number of spots of specific binding substances at different positions of the surface of a carrier such as a slide glass plate at high density and hybridizing them with a substance derived from a living organism and labeled with a labeling substance.

In addition, a macro-array analyzing system using a radioactive labeling substance as a labeling substance has been further developed, which comprises the steps of using a spotting device to drop at different positions on the surface of a carrier such as a membrane filter or the like specific binding substances, which can specifically bind with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, thereby forming a number of independent spots, specifically binding the specific binding substance using a hybridization method or the like with a substance derived from a living organism such as a cell, virus, hormone, tumor marker, enzyme, antibody, antigen, abzyme, other protein, a nuclear acid, cDNA, DNA or mRNA, which is gathered from a living organism by extraction, isolation or the like or is further subjected to chemical processing, chemical modification or the like and which is labeled with a radioactive labeling substance, thereby forming a macro-array, superposing the macro-array and a stimulable phosphor sheet formed with a stimulable phosphor layer, exposing the stimulable phosphor layer to a radioactive labeling substance, irradiating the stimulable phosphor layer with a stimulating ray to excite the stimulable phosphor, photoelectrically detecting the stimulated emission released from the stimulable phosphor to produce biochemical analysis data, and analyzing the substance derived from a living organism.

However, in the macro-array analyzing system using a radioactive labeling substance as a labeling substance, when the stimulable phosphor layer is exposed to a radioactive labeling substance, since radiation energy of the radioactive labeling substance contained in spots formed on the surface of a carrier such as a membrane filter is very large, electron beams released from the radioactive labeling substance contained in the individual spots are scattered in the carrier such as a membrane filter, thereby impinging on regions of the stimulable phosphor layer that should be exposed to the radioactive labeling substance contained in neighboring spots, or electron beams released from the radioactive labeling substance contained in the individual spots are scattered and mixed with the electron beams released from the radioactive labeling substance contained in neighboring spots and then impinge on regions of the stimulable phosphor layer to generate noise in biochemical analysis data produced by photoelectrically detecting stimulated emission and to lower the accuracy of biochemical analysis when a substance derived from a living organism is analyzed by quantifying the radiation amount of each spot. The accuracy of biochemical analysis is markedly degraded when spots are formed closely to each other at high density.

In order to solve these problems by preventing noise caused by the scattering of electron beams released from radioactive labeling substance contained in neighboring spots, it is inevitably required to increase the distance between neighboring spots and this makes the density of the spots lower and the test efficiency lower.

Furthermore, in the field of biochemical analysis, it is often required to analyze a substance derived from a living organism by forming a plurality of spot-like regions containing specific binding substances at different positions on the surface of a carrier such as a membrane filter or the like, which can specifically bind with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, specifically binding, using a hybridization method or the like, the specific binding substances contained in the plurality of spot-like regions with a substance derived from a living organism labeled with a labeling substance which generates chemiluminescent emission when it contacts a chemiluminescent substrate, thereby selectively labeling the plurality of spot-like regions, causing the plurality of spot-like regions to come into contact with a chemiluminescent substrate, exposing a stimulable phosphor layer to chemiluminescent emission in the wavelength of visible light generated by the contact of the chemiluminescent substance and the labeling substance, thereby storing the energy of chemiluminescent emission in the stimulable phosphor layer, irradiating the stimulable phosphor layer with a stimulating ray, and photoelectrically detecting stimulated emission released from the stimulable phosphor layer, thereby effecting biochemical analysis. In this case, chemiluminescent emission released from any particular spot-like region is scattered in the carrier such as a membrane filter and mixed with chemiluminescent emission released from neighboring spot-like regions, thereby generating noise in biochemical analysis data produced by photoelectrically detecting chemiluminescent emission.

### SUMMARY OF THE INVENTION

It is therefore an object of the present invention to provide a biochemical analysis unit and a method for exposing a stimulable phosphor sheet using the same which can prevent noise caused by the scattering of electron beams released from a radioactive labeling substance from being generated in biochemical analysis data even in the case of forming a plurality of spot-like regions containing specific binding substances on the surface of a carrier at a high density, which can specifically bind with a substance derived from a living organism and whose sequence, base length, composition and the like are known, specifically binding the specific binding substances contained in the plurality of spot-like regions with a substance derived from a living organism labeled with a radioactive substance to selectively label the spot-like specific binding substances with the radioactive substance, thereby obtaining a biochemical analysis unit, superposing the thus obtained biochemical analysis unit and a stimulable phosphor layer, exposing the stimulable phosphor layer to the radioactive labeling substance, irradiating the stimulable phosphor layer with a stimulating ray to excite the stimulable phosphor, photoelectrically detecting the stimulated emission released from the stimulable phosphor layer to produce biochemical analysis data, and analyzing the substance derived from a living organism.

It is another object of the present invention to provide a biochemical analysis unit and a method for exposing a stimulable phosphor sheet using the same which can prevent noise caused by the scattering of chemiluminescent emission released from a plurality of spot-like regions of a biochemical analysis unit from being generated in biochemical analysis data even in the case of forming the plurality of spot-like regions containing specific binding substances on the surface of a carrier at a high density, which can specifically bind with a substance derived from a living organism and whose sequence, base length, composition and the like are known, specifically binding the specific binding substances contained in the plurality of spot-like regions with a substance derived from a living organism labeled with a labeling substance which generates chemiluminescent emission when it contacts a chemiluminescent substrate to produce a biochemical analysis unit, causing a chemiluminescent substrate to come into contact with the biochemical analysis unit, thereby causing the plurality of spot-like regions of the biochemical analysis unit to release chemiluminescent emission, holding the biochemical analysis unit whose plurality of spot-like regions are releasing chemiluminescent emission in close contact with a stimulable phosphor layer, exposing the stimulable phosphor layer to chemiluminescent emission, irradiating the stimulable phosphor layer with a stimulating ray, photoelectrically detecting stimulated emission released from the stimulable phosphor layer to produce biochemical analysis data, and analyzing the substance derived from a living organism.

The above other objects of the present invention can be accomplished by a biochemical analysis unit comprising a plurality of absorptive regions formed of absorptive material and spaced apart from each other and a plurality of isolating regions formed of a material capable of attenuating radiation energy and/or light energy for isolating the plurality of absorptive regions, the plurality of isolating regions being formed so that surfaces thereof lie outward of surfaces of the individual absorptive regions.

When the biochemical analysis unit according to the present invention is utilized, specific binding substances, which can specifically bind with a substance derived from a living organism and whose sequence, base length, composition and the like are known, are spotted in the plurality of absorption regions formed by absorptive material and a substance derived from a living organism and labeled with a radioactive substance is specifically bound, using a hybridization method or the like, with the specific binding substances contained in the plurality of absorptive regions, thereby selectively labeling the plurality of absorptive regions therewith. Next, a stimulable phosphor layer formed on a stimulable phosphor sheet is exposed to the radioactive labeling substance contained in the plurality of absorptive regions by facing the biochemical analysis unit toward the stimulable phosphor layer. Since the plurality of isolating regions for isolating the plurality of absorptive regions are made of a material capable of attenuating radiation energy and/or light energy, it is possible at this time to effectively prevent electron beams (β rays) released from the radioactive labeling substance contained in the plurality of absorptive regions from scattering in the plurality of isolating regions by superposing and keeping the biochemical analysis unit on the stimulable phosphor layer in such a manner that the plurality of isolating regions are in contact with the surface of the stimulable phosphor layer. Since it is therefore possible to selectively expose only the regions of the stimulable phosphor layer that the individual absorptive regions face to the electron beams (β rays) released from the radioactive labeling substance contained in the absorptive regions, it is possible to effectively prevent noise from being generated in biochemical analysis data produced by photoelectrically detecting stimulated emission released from the stimulable phosphor layer in response to the stimulation with a stimulating ray and to produce biochemical analysis data having a high quantitative accuracy.

Further, according to the present invention, since the plurality of isolating regions are formed so that the surfaces thereof lie outward of the surfaces of individual absorptive regions, electron beams (β rays) released from the radioactive labeling substance contained in the plurality of the absorptive regions are prevented by the collimation effect from broadening. Since it is therefore possible to selectively expose only the regions of the stimulable phosphor layer that the individual absorptive regions face to the electron beams (β rays) released from the radioactive labeling substance contained in the absorptive regions, it is possible to effectively prevent noise from being generated in biochemical analysis data produced by photoelectrically detecting stimulated emission released from the stimulable phosphor layer in response to the stimulation with a stimulating ray and to produce biochemical analysis data having a high quantitative accuracy.

Furthermore, according to the present invention, in the case of spotting specific binding substances on the plurality of absorptive regions formed of absorptive material, which can specifically bind with a substance derived from a living organism and whose sequence, base length, composition and the like are known, selectively labeling the the plurality of absorptive regions with a labeling substance which generates chemiluminescent emission when it contacts a chemiluminescent substrate to produce a biochemical analysis unit, causing a chemiluminescent substrate to come into contact with the plurality of absorptive regions of the biochemical analysis unit, thereby causing the plurality of absorptive regions of the biochemical analysis unit to release chemiluminescent emission, superposing the biochemical analysis unit whose plurality of absorptive regions are releasing chemiluminescent emission with a stimulable phosphor layer of a stimulable phosphor sheet in such a manner that the plurality of isolating regions abut against the surface of the stimulable phosphor layer formed on the stimulable phosphor sheet and exposing the stimulable phosphor layer of the stimulable phosphor sheet to chemiluminescent emission selectively released from the plurality of absorptive regions of the biochemical analysis unit, since the plurality of isolating regions are formed of a material capable of attenuating radiation energy and/or light energy, it is possible to prevent chemiluminescent emission released from the plurality of absorptive regions from being scattered in the plurality of isolating regions by holding the biochemical analysis unit in contact with the stimulable phosphor layer so that the plurality of isolating regions are in contact with the surface of the stimulable phosphor layer of the stimulable phosphor sheet. Therefore, since it is possible to selectively expose only the regions of the stimulable phosphor layer that the individual absorptive regions face to the chemiluminescent emission, it is possible to effectively prevent noise from being generated in biochemical analysis data produced by photoelectrically detecting stimulated emission released from the stimulable phosphor layer in response to the stimulation with a stimulating ray and to produce biochemical analysis data having a high quantitative accuracy.

Moreover, according to the present invention, since the plurality of isolating regions are formed so that the surfaces thereof lie outward of the surfaces of individual absorptive regions, chemiluminescent emission released from the plurality of the absorptive regions are prevented by the collimation effect from broadening. Since it is therefore possible to selectively expose only the regions of the stimulable phosphor layer that the individual absorptive regions face to the chemiluminescent emission, it is possible to effectively prevent noise from being generated in biochemical analysis data produced by photoelectrically detecting stimulated emission released from the stimulable phosphor layer in response to the stimulation with a stimulating ray and to produce biochemical analysis data having a high quantitative accuracy.

The above and other objects of the present invention can be also accomplished by a method for exposing a stimulable phosphor sheet comprising the step of superposing a biochemical analysis unit including a plurality of absorptive regions formed of absorptive material and spaced apart from each other and a plurality of isolating regions formed of a material capable of attenuating radiation energy for isolating the plurality of absorptive regions, the plurality of isolating regions being formed so that surfaces thereof lie outward of surfaces of individual absorptive regions, and prepared by spotting specific binding substances whose sequence, base length, composition and the like are known in the plurality of absorptive regions and specifically binding a substance derived from a living organism and labeled with a radioactive substance with the specific binding substances, thereby selectively labeling the plurality of absorptive regions, on a stimulable phosphor layer formed on a stimulable phosphor sheet in such a manner that the plurality of isolating regions are in contact with the stimulable phosphor layer formed on the stimulable phosphor sheet, thereby exposing the stimulable phosphor layer of the stimulable phosphor sheet to the radioactive labeling substance selectively contained in the plurality of absorptive regions of the biochemical analysis unit.

According to the present invention, a biochemical analysis unit including a plurality of absorptive regions formed of absorptive material and spaced apart from each other and a plurality of isolating regions formed of a material capable of attenuating radiation energy for isolating the plurality of absorptive regions, the plurality of isolating regions being formed so that surfaces thereof lie outward of surfaces of individual absorptive regions, and prepared by spotting specific binding substances whose sequence, base length, composition and the like are known in the plurality of absorptive regions and specifically binding a substance derived from a living organism and labeled with a radioactive substance with the specific binding substances, thereby selectively labeling the plurality of absorptive regions, is superposed on a stimulable phosphor layer formed on a stimulable phosphor sheet in such a manner that the plurality of isolating regions are in contact with the stimulable phosphor layer formed on the stimulable phosphor sheet, thereby exposing the stimulable phosphor layer of the stimulable phosphor sheet to the radioactive labeling substance selectively contained in the plurality of absorptive regions of the biochemical analysis unit. Therefore, during the exposure operation, it is possible to effectively prevent electron beams (β rays) released from the radioactive labeling substance contained in the plurality of absorptive regions from scattering in the plurality of isolating regions and electron beams (β rays) released from the radioactive labeling substance contained in the plurality of the absorptive regions are prevented by the collimation effect from broadening. Since it is therefore possible to selectively expose only the regions of the stimulable phosphor layer that the individual absorptive regions face to the electron beams (β rays) released from the radioactive labeling substance contained in the absorptive regions, it is possible to effectively prevent noise from being generated in biochemical analysis data produced by photoelectrically detecting stimulated emission released from the stimulable phosphor layer in response to the stimulation with a stimulating ray and to produce biochemical analysis data having a high quantitative accuracy.

In a preferred aspect of the present invention, the plurality of absorptive regions of the biochemical analysis unit are formed by charging absorptive material in a plurality of holes formed spaced apart from each other in a substrate made of a material capable of attenuating radiation energy and the plurality of isolating regions are constituted by the substrate.

When the biochemical analysis unit according to this preferred aspect of the present invention is utilized, specific binding substances, which can specifically bind with a substance derived from a living organism and whose sequence, base length, composition and the like are known, are spotted in the plurality of absorption regions formed by charging absorptive material in the plurality of holes formed spaced apart from each other in a substrate and a substance derived from a living organism and labeled with a radioactive substance is specifically bound, using a hybridization method or the like, with the specific binding substances contained in the plurality of absorptive regions, thereby selectively labeling the plurality of absorptive regions therewith. Next, a stimulable phosphor layer formed on a stimulable phosphor sheet is exposed to the radioactive labeling substance contained in the plurality of absorptive regions by facing the biochemical analysis unit toward the stimulable phosphor layer. Since the substrate of the biochemical analysis unit is made of a material capable of attenuating radiation energy, it is possible to effectively prevent electron beams (β rays) released from the radioactive labeling substance contained in the plurality of absorptive regions from scattering in the plurality of isolating regions by superposing and keeping the biochemical analysis unit on the stimulable phosphor layer in such a manner that the plurality of isolating regions are in contact with the surface of the stimulable phosphor layer. Since it is therefore possible to selectively expose only the regions of the stimulable phosphor layer that the individual absorptive regions face to the electron beams (β rays) released from the radioactive labeling substance contained in the absorptive regions, it is possible to effectively prevent noise from being generated in biochemical analysis data produced by photoelectrically detecting stimulated emission released from the stimulable phosphor layer in response to the stimulation with a stimulating ray and to produce biochemical analysis data having a high quantitative accuracy.

Further, according to this preferred aspect of the present invention, since the plurality of absorptive regions are formed by charging absorptive material in the plurality of holes formed in the substrate and the plurality of absorptive regions are formed so that the surface of the substrate lies outward of the surfaces of the individual absorptive regions, electron beams (β rays) released from the radioactive labeling substance contained in the plurality of the absorptive regions are prevented by the collimation effect from broadening. Since it is therefore possible to selectively expose only the regions of the stimulable phosphor layer that the individual absorptive regions face to the electron beams (β rays) released from the radioactive labeling substance contained in each of the absorptive regions, it is possible to effectively prevent noise from being generated in biochemical analysis data produced by photoelectrically detecting stimulated emission released from the stimulable phosphor layer in response to the stimulation with a stimulating ray and to produce biochemical analysis data having a high quantitative accuracy.

The above and other objects of the present invention can be also accomplished by a method for exposing a stimulable phosphor sheet comprising the step of causing a biochemical analysis unit including a plurality of absorptive regions formed of absorptive material and spaced apart from each other and a plurality of isolating regions formed of a material capable of attenuating light energy for isolating the plurality of absorptive regions, the plurality of isolating regions being formed so that surfaces thereof lie outward of surfaces of individual absorptive regions, and prepared by spotting specific binding substances whose sequence, base length, composition and the like are known in the plurality of absorptive regions and specifically binding a substance derived from a living organism and labeled with a labeling substance which generates chemiluminescent emission when it contacts a chemiluminescent substrate with the specific binding substances, thereby selectively labeling the plurality of absorptive regions, to come into contact with a chemiluminescent substrate, thereby causing the plurality of absorptive regions to release chemiluminescent emission, superposing the biochemical analysis unit whose plurality of absorptive regions are releasing chemiluminescent emission with a stimulable phosphor layer of a stimulable phosphor sheet in such a manner that the plurality of isolating regions abut against the surface of the stimulable phosphor layer formed on the stimulable phosphor sheet and exposing the stimulable phosphor layer of the stimulable phosphor sheet to chemiluminescent emission selectively released from the plurality of absorptive regions of the biochemical analysis unit.

According to the present invention, since a method for exposing a stimulable phosphor sheet comprising the step of causing a biochemical analysis unit including a plurality of absorptive regions formed of absorptive material and spaced apart from each other and a plurality of isolating regions formed of a material capable of attenuating light energy for isolating the plurality of absorptive regions, the plurality of isolating regions being formed so that surfaces thereof lie outward of surfaces of individual absorptive regions, and prepared by spotting specific binding substances whose sequence, base length, composition and the like are known in the plurality of absorptive regions and specifically binding a substance derived from a living organism and labeled with a labeling substance which generates chemiluminescent emission when it contacts a chemiluminescent substrate with the specific binding substances, thereby selectively labeling the plurality of absorptive regions, to come into contact with a chemiluminescent substrate, thereby causing the plurality of absorptive regions to release chemiluminescent emission, superposing the biochemical analysis unit whose plurality of absorptive regions are releasing chemiluminescent emission with a stimulable phosphor layer of a stimulable phosphor sheet in such a manner that the plurality of isolating regions abut against the surface of the stimulable phosphor layer formed on the stimulable phosphor sheet and exposing the stimulable phosphor layer of the stimulable phosphor sheet to chemiluminescent emission selectively released from the plurality of absorptive regions of the biochemical analysis unit, when the stimulable phosphor layer of the stimulable phosphor sheet is exposed to chemiluminescent emission released from the plurality of absorptive regions of the biochemical analysis unit, it is possible to effectively prevent chemiluminescent emission from being scattered in the plurality of isolating regions and from broadening by the collimation effect. Therefore, since it is possible to selectively expose only the regions of the stimulable phosphor layer that the individual absorptive regions face to the chemiluminescent emission, it is possible to effectively prevent noise from being generated in biochemical analysis data produced by photoelectrically detecting stimulated emission released from the stimulable phosphor layer in response to the stimulation with a stimulating ray and to produce biochemical analysis data having a high quantitative accuracy.

In a preferred aspect of the present invention, the plurality of absorptive regions of the biochemical analysis unit are formed by charging absorptive material in a plurality of holes formed spaced apart from each other in a substrate made of a material capable of attenuating light energy and the plurality of isolating regions are constituted by the substrate.

According to this preferred aspect of the present invention, in the case of causing a biochemical analysis unit prepared by spotting specific binding substances whose sequence, base length, composition and the like are known in the plurality of absorptive regions formed by charging absorptive material in a plurality of holes formed in the substrate and selectively labeling the plurality of absorptive regions with a labeling substance which generates chemiluminescent emission when it contacts a chemiluminescent substrate, to come into contact with a chemiluminescent substrate, thereby causing the plurality of absorptive regions to release chemiluminescent emission, superposing the biochemical analysis unit whose plurality of absorptive regions are releasing chemiluminescent emission with a stimulable phosphor layer of a stimulable phosphor sheet in such a manner that the plurality of isolating regions abut against the surface of the stimulable phosphor layer formed on the stimulable phosphor sheet and exposing the stimulable phosphor layer of the stimulable phosphor sheet to chemiluminescent emission selectively released from the plurality of absorptive regions of the biochemical analysis unit, since the substrate of the biochemical analysis unit is formed of a material capable of attenuating light energy, it is possible to prevent chemiluminescent emission released from the plurality of absorptive regions from being scattered in the substrate of the biochemical analysis unit. Therefore, since it is possible to selectively expose only the regions of the stimulable phosphor layer that the individual absorptive regions face to the chemiluminescent emission, it is possible to effectively prevent noise from being generated in biochemical analysis data produced by photoelectrically detecting stimulated emission released from the stimulable phosphor layer in response to the stimulation with a stimulating ray and to produce biochemical analysis data having a high quantitative accuracy.

Further, according to this preferred aspect of the present invention, since the plurality of absorptive regions are formed by charging absorptive material in a plurality of holes formed in the substrate and the surface of the substrate lies outward of the surfaces of individual absorptive regions, chemiluminescent emission released from the plurality of the absorptive regions are prevented by the collimation effect from broadening. Since it is therefore possible to selectively expose only the regions of the stimulable phosphor layer that the individual absorptive regions face to the chemiluminescent emission, it is possible to effectively prevent noise from being generated in biochemical analysis data produced by photoelectrically detecting stimulated emission released from the stimulable phosphor layer in response to the stimulation with a stimulating ray and to produce biochemical analysis data having a high quantitative accuracy.

In a preferred aspect of the present invention, the biochemical analysis unit is prepared by specifically binding a substance derived from a living organism and labeled with a labeling substance which generates chemiluminescent emission when it contacts a chemiluminescent substrate with the specific binding substances contained in the plurality of absorptive regions, thereby selectively labeling the plurality of absorptive regions.

In another preferred aspect of the present invention, the biochemical analysis unit is prepared by selectively binding a substance derived from a living organism and labeled with hapten with the specific binding substances contained in the plurality of absorptive regions, binding an antibody for the hapten labeled with an enzyme having a property to generate chemiluminescent emission when it contacts a chemiluminescent substrate with the hapten by an antigen-antibody reaction, thereby selectively labeling the plurality of absorptive regions with the enzyme.

In the present invention, illustrative examples of the combination of hapten and antibody include digoxigenin and anti-digoxigenin antibody, theophylline and anti-theophylline antibody, fluorosein and anti-fluorosein antibody, and the like. Further, the combination of biotin and avidin, antigen and antibody may be utilized instead of the combination of hapten and antibody.

In a preferred aspect of the present invention, the substance derived from a living organism is specifically bound with specific binding substances by a reaction selected from a group consisting of hybridization, antigen-antibody reaction and receptor-ligand reaction.

In a further preferred aspect of the present invention, the plurality of absorptive regions of the biochemical analysis unit are formed by charging absorptive material in a plurality of through-holes formed spaced apart from each other in a substrate made of a material capable of attenuating radiation energy and/or light energy and the plurality of isolating regions are constituted by the substrate.

In another preferred aspect of the present invention, the plurality of absorptive regions of the biochemical analysis unit are formed by charging absorptive material in a plurality of recesses formed spaced apart from each other in a substrate made of a material capable of attenuating radiation energy and/or light energy and the plurality of isolating regions are constituted by the substrate.

In a preferred aspect of the present invention, the plurality of isolating regions of the biochemical analysis unit are formed in such a manner that the surfaces thereof lie outward of the surfaces of the respective absorptive regions by 0.5 to 100 times the maximum width of each of the absorptive regions.

In a further preferred aspect of the present invention, the plurality of isolating regions of the biochemical analysis unit are formed in such a manner that the surfaces thereof lie outward of the surfaces of the respective absorptive regions by 1 to 10 times the maximum width of each of the absorptive regions.

In a preferred aspect of the present invention, a plurality of absorptive regions having a substantially circular shape are formed in the biochemical analysis unit.

In a further preferred aspect of the present invention, the plurality of isolating regions are formed in such a manner that the surfaces thereof lie outward of the surfaces of the respective absorptive regions by 0.5 to 100 times the diameter of each of the absorptive regions.

In a further preferred aspect of the present invention, the plurality of isolating regions are formed in such a manner that the surfaces thereof lie outward of the surfaces of the respective absorptive regions by 1 to 10 times the diameter of each of the absorptive regions.

In another preferred aspect of the present invention, a plurality of absorptive regions having a substantially rectangular shape are formed in the biochemical analysis unit.

In a preferred aspect of the present invention, the plurality of absorptive regions are regularly formed in the biochemical analysis unit.

In a preferred aspect of the present invention, the biochemical analysis unit is formed with 10 or more absorptive regions.

In a further preferred aspect of the present invention, the biochemical analysis unit is formed with 50 or more absorptive regions.

In a further preferred aspect of the present invention, the biochemical analysis unit is formed with 100 or more absorptive regions.

In a further preferred aspect of the present invention, the biochemical analysis unit is formed with 500 or more absorptive regions.

In a further preferred aspect of the present invention, the biochemical analysis unit is formed with 1,000 or more absorptive regions.

In a further preferred aspect of the present invention, the biochemical analysis unit is formed with 5,000 or more absorptive regions.

In a further preferred aspect of the present invention, the biochemical analysis unit is formed with 10,000 or more absorptive regions.

In a further preferred aspect of the present invention, the biochemical analysis unit is formed with 50,000 or more absorptive regions.

In a further preferred aspect of the present invention, the substrate of the biochemical analysis unit is formed with 100,000 or more absorptive regions.

In a preferred aspect of the present invention, each of the plurality of absorptive regions formed in the biochemical analysis unit has a size of less than 5 mm².

In a further preferred aspect of the present invention, each of the plurality of absorptive regions formed in the biochemical analysis unit has a size of less than 1 mm².

In a further preferred aspect of the present invention, each of the plurality of absorptive regions formed in the biochemical analysis unit has a size of less than 0.5 mm².

In a further preferred aspect of the present invention, each of the plurality of absorptive regions formed in the biochemical analysis unit has a size of less than 0.1 mm².

In a further preferred aspect of the present invention, each of the plurality of absorptive regions formed in the biochemical analysis unit has a size of less than 0.05 mm².

In a further preferred aspect of the present invention, each of the plurality of absorptive regions formed in the biochemical analysis unit has a size of less than 0.01 mm².

In the present invention, the density of the absorptive regions formed in the biochemical analysis unit is determined depending upon the material for forming the plurality of isolating regions, the kind of electron beam released from a radioactive substance or the like.

In a preferred aspect of the present invention, the plurality of absorptive regions are formed in the biochemical analysis unit at a density of 10 or more per cm².

In a further preferred aspect of the present invention, the plurality of absorptive regions are formed in the biochemical analysis unit at a density of 50 or more per cm².

In a further preferred aspect of the present invention, the plurality of absorptive regions are formed in the biochemical analysis unit at a density of 100 or more per cm².

In a further preferred aspect of the present invention, the plurality of absorptive regions are formed in the biochemical analysis unit at a density of 500 or more per cm².

In a further preferred aspect of the present invention, the plurality of absorptive regions are formed in the biochemical analysis unit at a density of 1,000 or more per cm².

In a further preferred aspect of the present invention, the plurality of absorptive regions are formed in the biochemical analysis unit at a density of 5,000 or more per cm².

In a further preferred aspect of the present invention, the plurality of absorptive regions are formed in the biochemical analysis unit at a density of 10,000 or more per cm².

In a preferred aspect of the present invention, the isolating region of the biochemical analysis unit has a property of reducing the energy of radiation and/or the energy of light to ⅕ or less when the radiation and/or light travels in the isolating region by a distance equal to that between neighboring absorptive regions.

In a further preferred aspect of the present invention, the isolating region of the biochemical analysis unit has a property of reducing the energy of radiation and/or the energy of light to 1/10 or less when the radiation and/or light travels in the isolating region by a distance equal to that between neighboring absorptive regions.

In a further preferred aspect of the present invention, the isolating region of the biochemical analysis unit has a property of reducing the energy of radiation and/or the energy of light to 1/50 or less when the radiation and/or light travels in the isolating region by a distance equal to that between neighboring absorptive regions.

In a further preferred aspect of the present invention, the isolating region of the biochemical analysis unit has a property of reducing the energy of radiation and/or the energy of light to 1/100 or less when the radiation and/or light travels in the isolating region by a distance equal to that between neighboring absorptive regions.

In a further preferred aspect of the present invention, the isolating region of the biochemical analysis unit has a property of reducing the energy of radiation and/or the energy of light to 1/500 or less when the radiation and/or light travels in the isolating region by a distance equal to that between neighboring absorptive regions.

In a further preferred aspect of the present invention, the isolating region of the biochemical analysis unit has a property of reducing the energy of radiation and/or the energy of light to 1/1000 or less when the radiation and/or light travels in the isolating region by a distance equal to that between neighboring absorptive regions.

In the present invention, the material for forming an isolating region of a biochemical analysis unit is not particularly limited but may be of any type of inorganic compound material or organic compound material insofar as it can attenuate radiation energy and/or light energy. It is preferably formed of a metal material, a ceramic material or a plastic material.

In the present invention, illustrative examples of inorganic compound materials capable of attenuating radiation energy and/or light energy and preferably usable for forming an isolating region of a biochemical analysis unit include metals such as gold, silver, copper, zinc, aluminum, titanium, tantalum, chromium, iron, nickel, cobalt, lead, tin, selenium and the like; alloys such as brass, stainless steel, bronze and the like; silicon materials such as silicon, amorphous silicon, glass, quartz, silicon carbide, silicon nitride and the like; metal oxides such as aluminum oxide, magnesium oxide, zirconium oxide and the like; and inorganic salts such as tungsten carbide, calcium carbide, calcium sulfate, hydroxy apatite, gallium arsenide and the like. These may have either a monocrystal structure or a polycrystal sintered structure such as amorphous, ceramic or the like.

In the present invention, a high molecular compound is preferably used as an organic compound material capable of attenuating radiation energy and/or light energy. Illustrative examples of high molecular compounds preferably usable for forming an isolating region of a biochemical analysis unit include polyolefins such as polyethylene, polypropylene and the like; acrylic resins such as polymethyl methacrylate, polybutylacrylate/polymethyl methacrylate copolymer and the like; polyacrylonitrile; polyvinyl chloride; polyvinylidene chloride; polyvinylidene fluoride; polytetrafluoroethylene; polychlorotrifuluoroethylene; polycarbonate; polyesters such as polyethylene naphthalate, polyethylene terephthalate and the like; nylons such as nylon-6, nylon-6,6, nylon-4, 10 and the like; polyimide; polysulfone; polyphenylene sulfide; silicon resins such as polydiphenyl siloxane and the like; phenol resins such as novolac and the like; epoxy resin; polyurethane; polystyrene, butadiene-styrene copolymer; polysaccharides such as cellulose, acetyl cellulose, nitrocellulose, starch, calcium alginate, hydroxypropyl methyl cellulose and the like; chitin; chitosan; urushi (Japanese lacquer); polyamides such as gelatin, collagen, keratin and the like; and copolymers of these high molecular materials. These may be a composite compound, and metal oxide particles, glass fiber or the like may be added thereto as occasion demands. Further, an organic compound material may be blended therewith.

Since the capability of attenuating radiation energy generally increases as specific gravity increases, the isolating region of the biochemical analysis unit is preferably formed of a compound material or a composite material having specific gravity of 1.0 g/cm³ or more and more preferably formed of a compound material or a composite material having specific gravity of 1.5 g/cm³ to 23 g/cm³.

Since the capability of attenuating light energy generally increases as scattering and/or absorption of light increases, the isolating region of the biochemical analysis unit preferably has absorbance of 0.3 per cm (thickness) or more and more preferably has absorbance of 1 per cm (thickness) or more. The absorbance can be determined by placing an integrating sphere immediately behind a plate-like member having a thickness of T cm, measuring an amount A of transmitted light at a wavelength of probe light or emission light used for measurement by a spectrophotometer, and calculating A/T. In the present invention, a light scattering substance or a light absorbing substance may be added to the isolating region of the biochemical analysis unit in order to improve the capability of attenuating light energy. Particles of a material different from a material forming the isolating region of the biochemical analysis unit may be preferably used as a light scattering substance and a pigment or dye may be preferably used as a light absorbing substance.

In a preferred aspect of the present invention, the substrate of the biochemical analysis unit is formed of a flexible material.

According to this preferred aspect of the present invention, since the substrate of the biochemical analysis unit is formed of a flexible material, the biochemical analysis unit can be bent and be brought into contact with a reaction solution such as a hybridization reaction solution, thereby specifically binding specific binding substances with a substance derived from a living organism. Therefore, specific binding substances and a substance derived from a living organism can be specifically bound with each other in a desired manner using a small amount of a reaction solution such as a hybridization reaction solution.

In the present invention, a porous material or a fiber material may be preferably used as the absorptive material for forming the absorptive regions. The absorptive substrate may be formed by combining a porous material and a fiber material.

In the present invention, a porous material for forming the absorptive regions may be any type of an organic material or an inorganic material and may be an organic/inorganic composite material.

In the present invention, an organic porous material used for forming the absorptive regions is not particularly limited but a carbon porous material such as an activated carbon or a porous material capable of forming a membrane filter is preferably used. Illustrative examples of porous materials capable of forming a membrane filter include nylons such as nylon-6, nylon-6,6, nylon-4,10; cellulose derivatives such as nitrocellulose, acetyl cellulose, butyric-acetyl cellulose; collagen; alginic acids such as alginic acid, calcium alginate, alginic acid/poly-L-lysine polyionic complex; polyolefins such as polyethylene, polypropylene; polyvinyl chloride; polyvinylidene chloride; polyfluoride such as polyvinylidene fluoride, polytetrafluoride; and copolymers or composite materials thereof.

In the present invention, an inorganic porous material used for forming the absorptive regions is not particularly limited. Illustrative examples of inorganic porous materials preferably usable in the present invention include metals such as platinum, gold, iron, silver, nickel, aluminum and the like; metal oxides such as alumina, silica, titania, zeolite and the like; metal salts such as hydroxy apatite, calcium sulfate and the like; and composite materials thereof.

In the present invention, a fiber material used for forming the absorptive regions is not particularly limited. Illustrative examples of fiber materials preferably usable in the present invention include nylons such as nylon-6, nylon-6,6, nylon-4,10; and cellulose derivatives such as nitrocellulose, acetyl cellulose, butyric-acetyl cellulose.

In the present invention, the stimulable phosphor usable for storing radiation energy may be of any type insofar as it can store radiation energy or electron beam energy and can be stimulated by an electromagnetic wave to release the radiation energy or the electron beam energy stored therein in the form of light. More specifically, preferably employed stimulable phosphors include alkaline earth metal fluorohalide phosphors (Ba₁₋ₓ, M²⁺ₓ)FX:yA (where M²⁺ is at least one alkaline earth metal selected from the group consisting of Mg, Ca, Sr, Zn and Cd; X is at least one element selected from the group consisting of Cl, Br and I, A is at least one element selected from the group consisting of Eu, Tb, Ce, Tm, Dy, Pr, Ho, Nd, Yb and Er; x is equal to or greater than 0 and equal to or less than 0.6 and y is equal to or greater than 0 and equal to or less than 0.2) disclosed in U.S. Pat. No. 4,239,968, alkaline earth metal fluorohalide phosphors SrFX:Z (where X is at least one halogen selected from the group consisting of Cl, Br and I; Z is at least one of Eu and Ce) disclosed in Japanese Patent Application Laid Open No. 2-276997, europium activated complex halide phosphors BaFXxNaX′:aEu²⁺ (where each of X or X′ is at least one halogen selected from the group consisting of Cl, Br and I; x is greater than 0 and equal to or less than 2; and y is greater than 0 and equal to or less than 0.2) disclosed in Japanese Patent Application Laid Open No. 59-56479, cerium activated trivalent metal oxyhalide phosphors MOX:xCe (where M is at least one trivalent metal selected from the group consisting of Pr, Nd, Pm, Sm, Eu, Tb, Dy, Ho, Er, Tm, Yb and Bi; X is at least one halogen selected from the group consisting of Br and I; and x is greater than 0 and less than 0.1) disclosed in Japanese Patent Application laid Open No. 58-69281, cerium activated rare earth oxyhalide phosphors LnOX:xCe (where Ln is at least one rare earth element selected from the group consisting of Y, La, Gd and Lu; X is at least one halogen selected from the group consisting of Cl, Br and I; and x is greater than 0 and equal to or less than 0.1) disclosed in U.S. Pat. No. 4,539,137, and europium activated complex halide phosphors M^{II}FXaM^{I}X′bM′^{II}X″₂cM^{III}X′″₃xA:yEu²⁺ (where M^{II} is at least one alkaline earth metal selected from the group consisting of Ba, Sr and Ca; M^{I} is at least one alkaline metal selected from the group consisting of Li, Na, K, Rb and Cs; M′^{II} is at least one divalent metal selected from the group consisting of Be and Mg; M^{III} is at least one trivalent metal selected from the group consisting of Al, Ga, In and Ti; A is at least one metal oxide; X is at least one halogen selected from the group consisting of Cl, Br and I; each of X′, X″ and X′″ is at least one halogen selected from the group consisting of F, Cl, Br and I; a is equal to or greater than 0 and equal to or less than 2; b is equal to or greater than 0 and equal to or less than 10⁻²; c is equal to or greater than 0 and equal to or less than 10⁻²; a+b+c is equal to or greater than 10⁻²; x is greater than 0 and equal to or less than 0.5; and y is greater than 0 and equal to or less than 0.2) disclosed in U.S. Pat. No. 4,962,047.

In the present invention, the stimulable phosphor usable for storing the energy of chemiluminescence emission may be of any type insofar as it can store the energy of light in the wavelength band of visible light and can be stimulated by an electromagnetic wave to release in the form of light the energy of light in the wavelength band of visible light stored therein. More specifically, preferably employed stimulable phosphors include at least one selected from the group consisting of metal halophosphates, rare-earth-activated sulfide-host phosphors, aluminate-host phosphors, silicate-host phosphors, fluoride-host phosphors and mixtures of two, three or more of these phosphors. Among them, rare-earth-activated sulfide-host phosphors are more preferable and, particularly, rare-earth-activated alkaline earth metal sulfide-host phosphors disclosed in U.S. Pat. Nos. 5,029,253 and 4,983,834, zinc germanate such as Zn₂GeO₄:Mn, V; Zn₂GeO₄:Mn disclosed in Japanese Patent Application Laid Open No. 2001-131545, alkaline-earth aluminate such as Sr₄Al₁₄O₂₅:Ln (wherein Ln is a rare-earth element) disclosed in Japanese Patent Application Laid Open No. 2001-123162, Y_{0.8}Lu_{1.2}SiO₅:Ce, Zr; GdOCl:Ce disclosed in Japanese Patent Publication No. 6-31904 and the like are most preferable.

The above and other objects and features of the present invention will become apparent from the following description made with reference to the accompanying drawings.

### BRIEF DESCRIPTION OF THE DRAWINGS

FIG. 1 is a schematic perspective view showing a biochemical analysis unit which is a preferred embodiment of the present invention.

FIG. 2 is a schematic partial cross-sectional view of a biochemical analysis unit.

FIG. 3 is a schematic front view showing a spotting device.

FIG. 4 is a schematic front view showing a hybridization reaction vessel.

FIG. 5 is a schematic cross-sectional view showing a method for exposing a stimulable phosphor layer formed on a stimulable phosphor sheet by a radioactive labeling substance contained in absorptive regions.

FIG. 6 is a schematic view showing one example of a scanner.

FIG. 7 is a schematic perspective view showing details in the vicinity of a photomultiplier.

FIG. 8 is a schematic cross-sectional view taken along a line A-A in FIG. 7.

FIG. 9 is a schematic cross-sectional view taken along a line B-B in FIG. 7.

FIG. 10 is a schematic cross-sectional view taken along a line C-C in FIG. 7.

FIG. 11 is a schematic cross-sectional view taken along a line D-D in FIG. 7.

FIG. 12 is a schematic plan view of a scanning mechanism of an optical head.

FIG. 13 is a block diagram of a control system, an input system and a drive system of a scanner shown in FIG. 6.

FIG. 14 is a schematic cross sectional view showing a biochemical analysis unit which is another preferred embodiment of the present invention.

### DESCRIPTION OF THE PREFERRED EMBODIMENTS

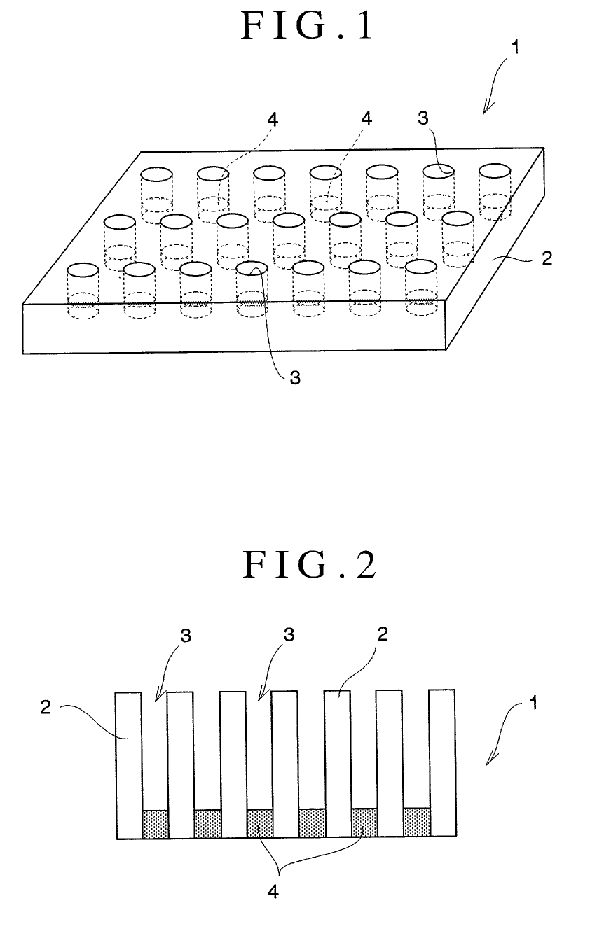

FIG. 1 is a schematic perspective view showing a biochemical analysis unit which is a preferred embodiment of the present invention.

As shown in FIG. 1, a biochemical analysis unit 1 includes a substrate 2 formed of metal such as stainless steel capable of attenuating radiation energy and light energy and having flexibility and formed with a number of substantially circular through-holes 3, and a number of absorptive regions 4 are dot-like formed by charging absorptive material such as nylon-6 in the through-holes 3.

Although not accurately shown in FIG. 1, in this embodiment, the through-holes 3 are formed in the substrate 2 so that substantially circular absorptive regions 4 having a size of about 0.07 cm² are regularly formed in the manner of a matrix of 120 columns×160 lines and, therefore, 19,200 absorptive regions 4 are formed.

FIG. 2 is a schematic partial cross-sectional view of the biochemical analysis unit 1.

As shown in FIG. 2, a number of absorptive regions 4 are formed by charging absorptive material 4 in the through-holes 3 formed in the substrate in such a manner that the surfaces of the absorptive regions 4 are lower than that of the substrate.

In this embodiment, each absorptive region 4 is formed by charging absorptive material 4 in the through-hole so that the difference between the surface of each absorptive region 4 and that of the substrate in the vertical direction, namely, the distance between the surface of each absorptive region 4 and that of the substrate is five times the diameter of the through-hole 3.

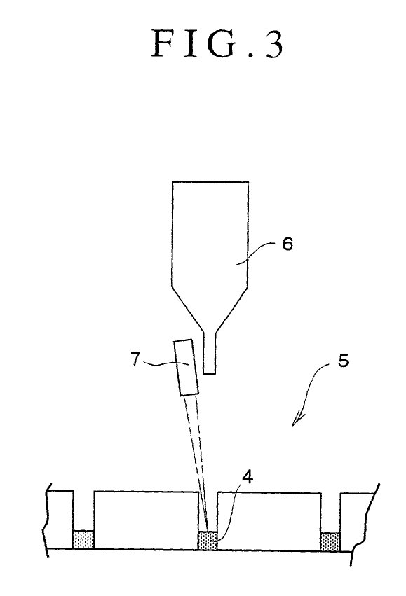

FIG. 3 is a schematic front view showing a spotting device.

As shown in FIG. 3, when biochemical analysis is performed, a solution containing specific binding substances such as a plurality of cDNAs whose sequences are known but are different from each other are spotted using a spotting device 5 onto a number of the absorptive regions 4 of the biochemical analysis unit 1 and the specific binding substances are fixed therein.

As shown in FIG. 3, the spotting device 5 includes an injector 6 for ejecting the solution of specific binding substances toward the biochemical analysis unit 1 and a CCD camera 7 and is constituted so that the solution of specific binding substances such as cDNAs are spotted from the injector 6 when the tip end portion of the injector 6 and the center of the absorptive region 4 into which a specific binding substance is to be spotted are determined to coincide with each other as a result of viewing them using the CCD camera, thereby ensuring that the solution of specific binding substances can be accurately spotted into a number of the absorptive regions 4 of the biochemical analysis unit 1.

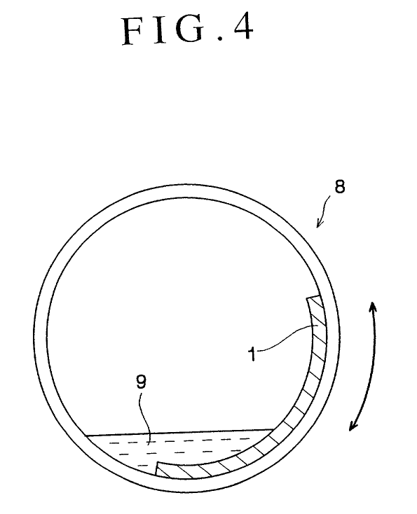

FIG. 4 is a schematic front view showing a hybridization reaction vessel.

As shown in FIG. 4, a hybridization reaction vessel 8 is formed cylindrically and accommodates a hybridization reaction solution 9 containing a substance derived from a living organism labeled with a labeling substance therein.

In this embodiment, a hybridization reaction solution 9 containing a substance derived from a living organism labeled with a radioactive labeling substance is prepared and accommodated in the hybridization reaction vessel 8.

When hybridization is to be performed, the biochemical analysis unit 1 containing specific binding substances such as a plurality of cDNAs spotted into a number of absorptive regions 4 in the through-holes 3 is accommodated in the hybridization reaction vessel 8. In this embodiment, since the substrate 2 is formed of a metal such as stainless steel having flexibility, as shown in FIG. 4, the biochemical analysis unit 1 can be bent and accommodated in the hybridization reaction vessel 8 along the inner wall surface thereof.

As shown in FIG. 4, the hybridization reaction vessel 8 is constituted so as to be rotatable about a shaft by a drive means (not shown) and since the biochemical analysis unit 1 is bent and accommodated in the hybridization vessel 8 along the inner wall surface thereof, even when the hybridization vessel 8 accommodates only a small amount of hybridization reaction solution 9, specific binding substances spotted in a number of the absorptive regions 4 can be selectively hybridized with a substance derived from a living organism labeled with a radioactive labeling substance and contained in the hybridization reaction solution 9 by rotating the hybridization reaction vessel 8.

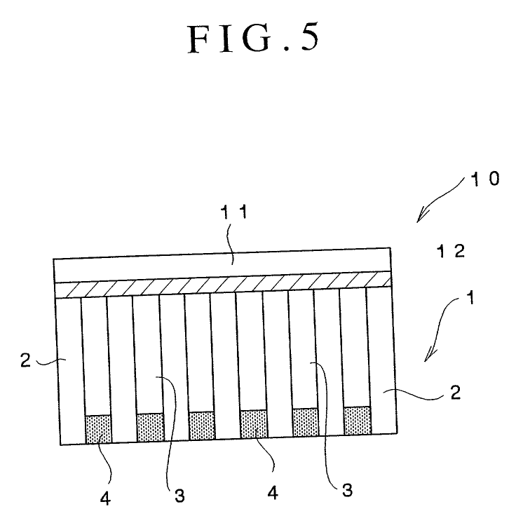

FIG. 5 is a schematic cross-sectional view showing a method for exposing a stimulable phosphor layer formed on a stimulable phosphor sheet to a radioactive labeling substance contained in a number of absorptive regions 4 of the biochemical analysis unit 1.

As shown in FIG. 5, when a stimulable phosphor layer 12 of a stimulable phosphor sheet 10 is to be exposed, the stimulable phosphor sheet 10 is superposed on the biochemical analysis unit 1 in such a manner that the stimulable phosphor layer 12 uniformly formed on one surface of a support 11 of the stimulable phosphor sheet 10 abuts against the surface of the substrate 2 of the biochemical analysis unit 1.

During the exposure operation, electron beams (β rays) are released from the radioactive labeling substance contained in the absorptive regions 4 of the biochemical analysis unit 1. However, since the substrate 2 of the biochemical analysis unit 1 is formed of a metal such as stainless steel capable of attenuating radiation energy, electron beams (β rays) released from the radioactive labeling substance contained in the absorptive regions 4 can be effectively prevented from scattering in the substrate 2 of the biochemical analysis unit 1. Further, since the absorptive region 4 is formed by charging absorptive material 4 in the through-hole so that the difference between the surface of each absorptive region 4 and that of the substrate in the vertical direction, namely, the distance between the surface of each absorptive region 4 and that of the substrate is five times the diameter of the through-hole 3, the electron beams (β rays) released from the radioactive labeling substance contained in each of the absorptive regions 4 are prevented by the collimation effect from broadening. Therefore, it is possible to selectively expose only the region of the stimulable phosphor layer 12 each of the absorptive regions 4 faces to the electron beams (β rays) released from the radioactive labeling substance contained in each of the absorptive regions 4.

In this manner, radiation data of a radioactive labeling substance are recorded in the stimulable phosphor layer 12 formed on the support 11 of the stimulable phosphor sheet 10.

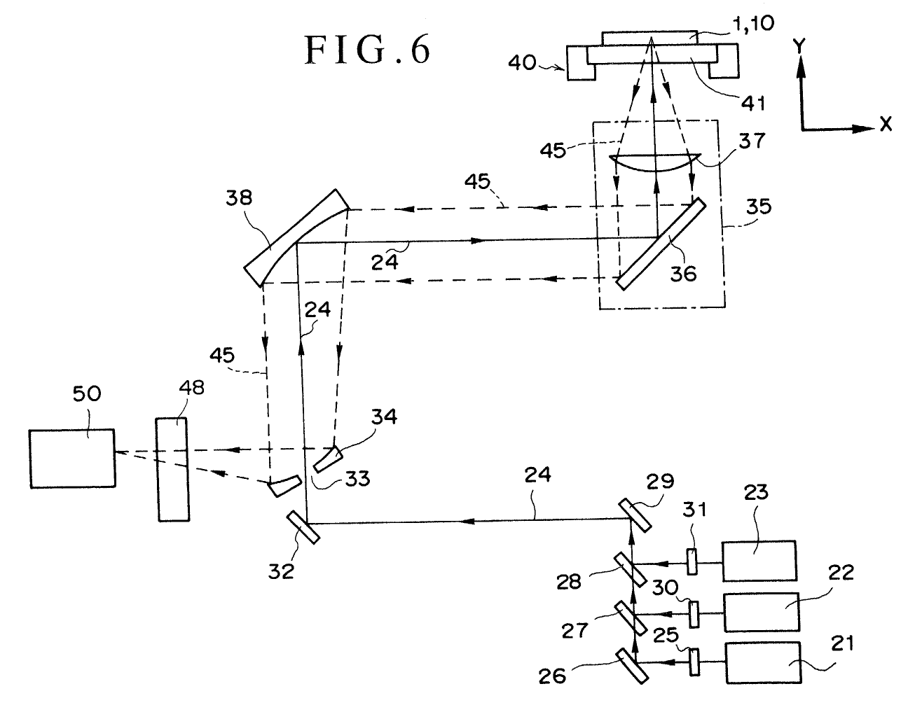

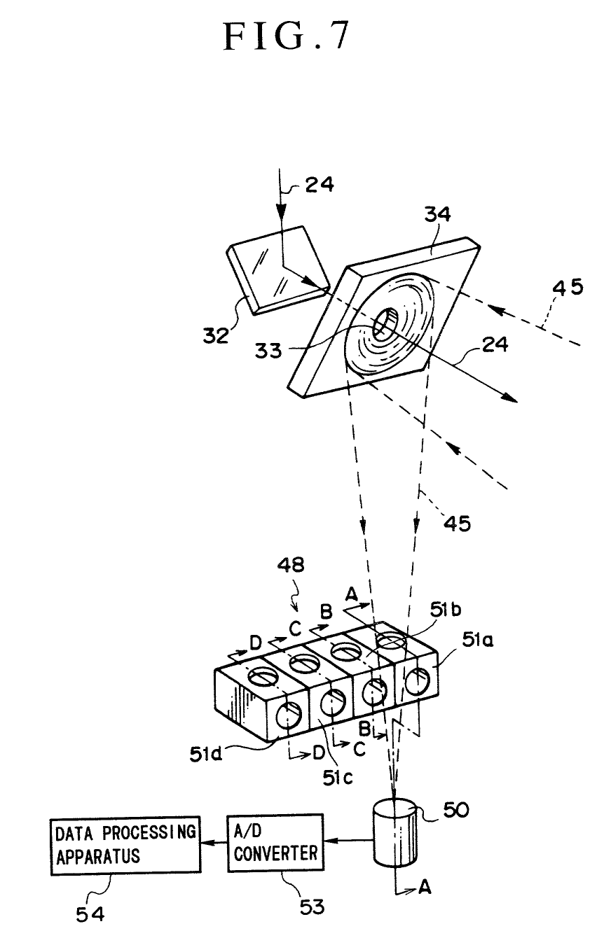

FIG. 6 is a schematic view showing one example of a scanner for reading radiation data of a radioactive labeling substance recorded in the stimulable phosphor layer 12 formed on the support 11 of the stimulable phosphor sheet 10 and producing biochemical analysis data, and FIG. 7 is a schematic perspective view showing details in the vicinity of a photomultiplier.

The scanner shown in FIG. 6 is constituted so as to read radiation data of a radioactive labeling substance recorded in the stimulable phosphor layer 12 formed on the support 11 of the stimulable phosphor sheet 10 and fluorescence data such as electrophoresis data of denatured DNA fragment s labeled with a fluorescent substance such as a fluorescent dye recorded in a gel support, a transfer support or the like and includes a first laser stimulating ray source 21 for emitting a laser beam having a wavelength of 640 nm, a second laser stimulating ray source 22 for emitting a laser beam having a wavelength of 532 nm and a third laser stimulating ray source 23 for emitting a laser beam having a wavelength of 473 nm.

In this embodiment, the first laser stimulating ray source 21 is constituted by a semiconductor laser beam source and the second laser stimulating ray source 22 and the third laser stimulating ray source 23 are constituted by a second harmonic generation element.

A laser beam 24 emitted from the first laser stimulating source 21 passes through a collimator lens 25, thereby being made a parallel beam, and is reflected by a mirror 26. A first dichroic mirror 27 for transmitting light having a wavelength of 640 nm but reflecting light having a wavelength of 532 nm and a second dichroic mirror 28 for transmitting light having a wavelength equal to and longer than 532 nm but reflecting light having a wavelength of 473 nm are provided in the optical path of the laser beam 24 emitted from the first laser stimulating ray source 21. The laser beam 24 emitted from the first laser stimulating ray source 21 and reflected by the mirror 26 passes through the first dichroic mirror 27 and the second dichroic mirror 28 and advances to a mirror 29.

On the other hand, the laser beam 24 emitted from the second laser stimulating ray source 22 passes through a collimator lens 30, thereby being made a parallel beam, and is reflected by the first dichroic mirror 27, thereby changing its direction by 90 degrees. The laser beam 24 then passes through the second dichroic mirror 28 and advances to the mirror 29.

Further, the laser beam 24 emitted from the third laser stimulating ray source 23 passes through a collimator lens 31, thereby being made a parallel beam, and is reflected by the second dichroic mirror 28, thereby changing its direction by 90 degrees. The laser beam 24 then advances to the mirror 29.

The laser beam 24 advancing to the mirror 29 is reflected by the mirror 29 and advances to a mirror 32 to be reflected thereby.

A perforated mirror 34 formed with a hole 33 at the center portion thereof is provided in the optical path of the laser beam 24 reflected by the mirror 32. The laser beam 24 reflected by the mirror 32 passes through the hole 33 of the perforated mirror 34 and advances to a concave mirror 38.

The laser beam 24 advancing to the concave mirror 38 is reflected by the concave mirror 38 and enters an optical head 35.

The optical head 35 includes a mirror 36 and an aspherical lens 37. The laser beam 24 entering the optical head 35 is reflected by the mirror 36 and condensed by the aspherical lens 37 onto the stimulable phosphor sheet 10, or a gel support or a transfer support placed on the glass plate 41 of a stage 40.

When the laser beam 24 impinges on the stimulable phosphor layer 12 of the stimulable phosphor 10, stimulable phosphor contained in the stimulable phosphor layer 12 formed on the support 11 of the stimulable phosphor 10 is excited, thereby releasing stimulated emission 45. On the other hand, when the laser beam 24 impinges on the gel support or the transfer support, a fluorescent substance such as a fluorescent dye contained in the gel support or the transfer support is excited, thereby releasing fluorescence emission 45.

The stimulated emission 45 released from the stimulable phosphor layer 12 of the stimulable phosphor 10 or the fluorescence emission 45 released from the gel support or the transfer support is condensed onto the mirror 36 by the aspherical lens 37 provided in the optical head 35 and reflected by the mirror 36 on the side of the optical path of the laser beam 24, thereby being made a parallel beam to advance to the concave mirror 38.

The stimulated emission 45 or the fluorescence emission 45 advancing to the concave mirror 38 is reflected by the concave mirror 38 and advances to the perforated mirror 34.

As shown in FIG. 7, the stimulated emission 45 or the fluorescence emission 45 advancing to the perforated mirror 34 is reflected downward by the perforated mirror 34 formed as a concave mirror and advances to a filter unit 48, whereby light having a predetermined wavelength is cut. The stimulated emission 45 or the fluorescence emission 45 then impinges on a photomultiplier 50, thereby being photoelectrically detected.

As shown in FIG. 7, the filter unit 48 is provided with four filter members 51a, 51b, 51c and 51d and is constituted to be laterally movable in FIG. 7 by a motor (not shown).

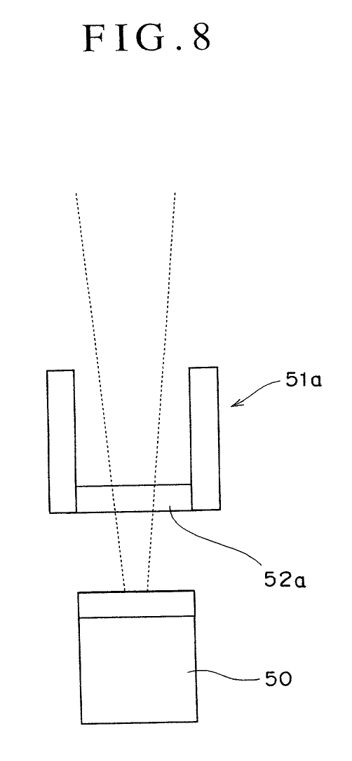

FIG. 8 is a schematic cross-sectional view taken along a line A-A in FIG. 7.

As shown in FIG. 8, the filter member 51a includes a filter 52a and the filter 52a is used for reading fluorescence emission 45 by stimulating a fluorescent substance such as a fluorescent dye contained in a gel support or a transfer support using the first laser stimulating ray source 21 and has a property of cutting off light having a wavelength of 640 nm but transmitting light having a wavelength longer than 640 nm.

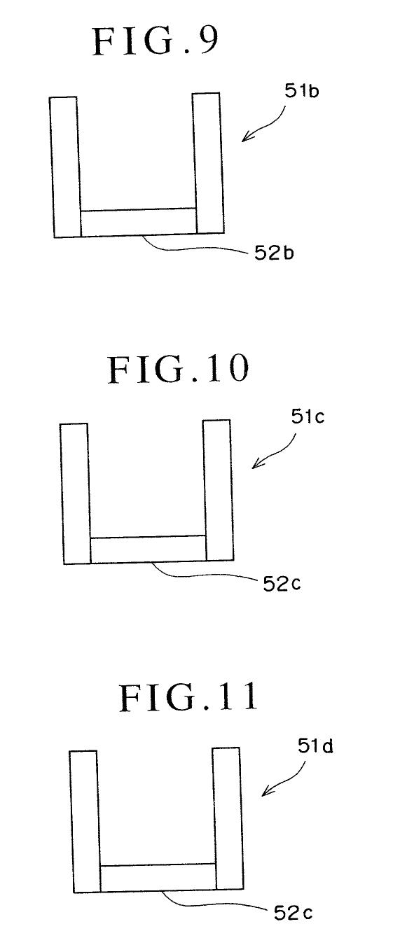

FIG. 9 is a schematic cross-sectional view taken along a line B-B in FIG. 7.

As shown in FIG. 9, the filter member 51b includes a filter 52b and the filter 52b is used for reading fluorescence emission 45 by stimulating a fluorescent substance such as a fluorescent dye contained in a gel support or a transfer support using the second laser stimulating ray source 22 and has a property of cutting off light having a wavelength of 532 nm but transmitting light having a wavelength longer than 532 nm.

FIG. 10 is a schematic cross-sectional view taken along a line C-C in FIG. 7.

As shown in FIG. 10, the filter member 51c includes a filter 52c and the filter 52c is used for reading fluorescence emission 45 by stimulating a fluorescent substance such as a fluorescent dye contained in a gel support or a transfer support using the third laser stimulating ray source 23 and has a property of cutting off light having a wavelength of 473 nm but transmitting light having a wavelength longer than 473 nm.

FIG. 11 is a schematic cross-sectional view taken along a line D-D in FIG. 7.

As shown in FIG. 11, the filter member 51d includes a filter 52d and the filter 52d is used for reading stimulated emission released from stimulable phosphor contained in the stimulable phosphor layer 12 formed on the support 11 of the stimulable phosphor sheet 10 upon being stimulated using the first laser stimulating ray source 1 and has a property of transmitting only light having a wavelength corresponding to that of stimulated emission emitted from stimulable phosphor but cutting off light having a wavelength of 640 nm.

Therefore, in accordance with the kind of a stimulating ray source to be used, one of these filter members 51a, 51b, 51c, 51d is selectively positioned in front of the photomultiplier 50, thereby enabling the photomultiplier 50 to photoelectrically detect only light to be detected.

The analog data produced by photoelectrically detecting light with the photomultiplier 50 are converted by an A/D converter 53 into digital data and the digital data are fed to a data processing apparatus 54.

Although not shown in FIG. 6, the optical head 35 is constituted to be movable by a scanning mechanism in the X direction and the Y direction in FIG. 6 so that the whole surface of the stimulable phosphor layer 12 formed on the support 11 of the stimulable phosphor sheet 10 or the whole surface of the gel support or the transfer support can be scanned by the laser beam 24.

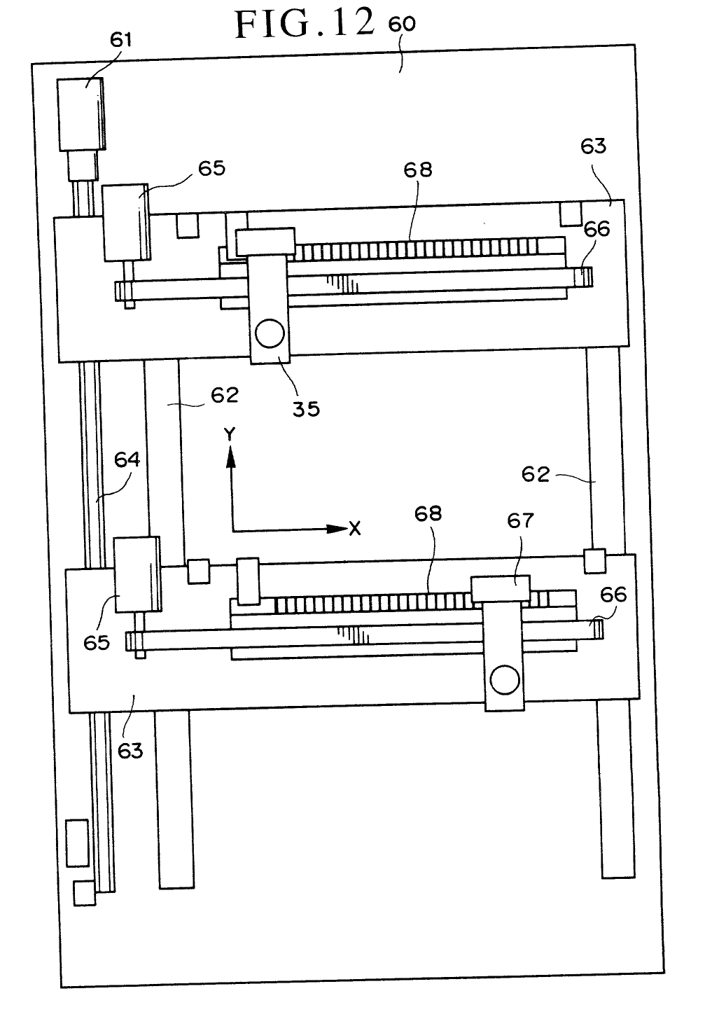

FIG. 12 is a schematic plan view showing the scanning mechanism of the optical head 35. In FIG. 12, optical systems other than the optical head 35 and the paths of the laser beam 24 and stimulated emission 45 or fluorescence emission 45 are omitted for simplification.

As shown in FIG. 12, the scanning mechanism of the optical head 35 includes a base plate 60, and a sub-scanning pulse motor 61 and a pair of rails 62, 62 are fixed on the base plate 60. A movable base plate 63 is further provided so as to be movable in the sub-scanning direction indicated by an arrow Y in FIG. 12.

The movable base plate 63 is formed with a threaded hole (not shown) and a threaded rod 64 rotated by the sub-scanning pulse motor 61 is engaged with the inside of the hole.

A main scanning pulse motor 65 is provided on the movable base plate 63. The main scanning pulse motor 65 is adapted for intermittently driving an endless belt 66 by the pitch equal to the distance between the neighboring absorptive regions 4 formed in the biochemical analysis unit 1. The optical head 35 is fixed to the endless belt 66 and when the endless belt 66 is driven by the main scanning pulse motor 65, the optical head 35 is moved in the main scanning direction indicated by an arrow X in FIG. 12. In FIG. 12, the reference numeral 67 designates a linear encoder for detecting the position of the optical head 35 in the main scanning direction and the reference numeral 68 designates slits of the linear encoder 67.

Therefore, the optical head 35 is moved in the X direction and the Y direction in FIG. 12 by driving the endless belt 66 in the main scanning direction by the main scanning pulse motor 65 and intermittently moving the movable base plate 63 in the sub-scanning direction by the sub-scanning pulse motor 61, thereby scanning the whole surface of the stimulable phosphor layer 12 formed on the support 11 of the stimulable phosphor sheet 10 or all of the absorptive regions 4 formed in the biochemical analysis unit 1 with the laser beam 24.

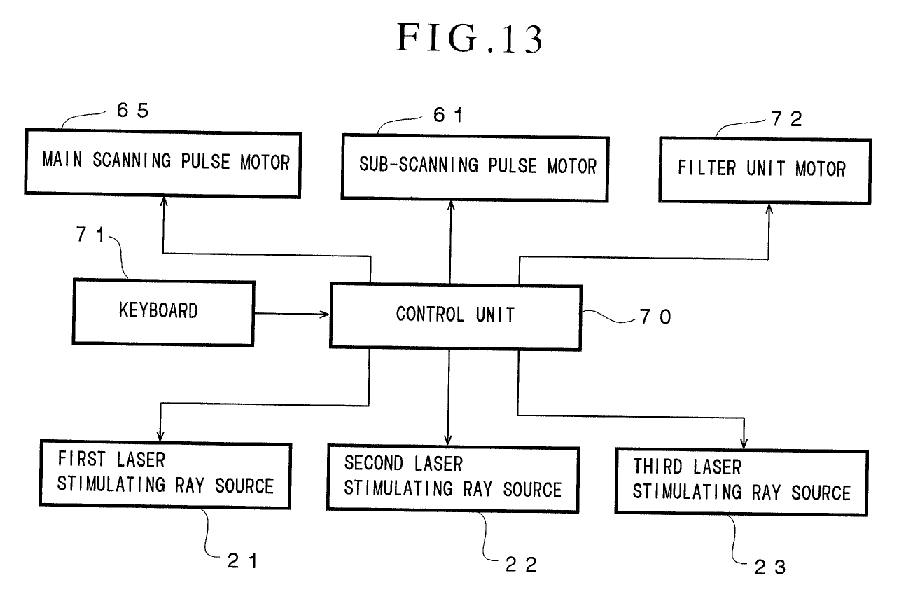

FIG. 13 is a block diagram of a control system, an input system and a drive system of the scanner shown in FIG. 6.

As shown in FIG. 13, the control system of the scanner includes a control unit 70 for controlling the whole operation of the scanner and the input system of the scanner includes a keyboard 71 which can be operated by a user and through which various instruction signals can be input.

As shown in FIG. 13, the drive system of the scanner includes the main scanning pulse motor 65 for moving the optical head 35 in the main scanning direction, the sub-scanning pulse motor 61 for moving the optical head 35 in the sub-scanning direction and a filter unit motor 72 for moving the filter unit 48 provided with the four filter members 51a, 51b, 51c and 51d.

The control unit 70 is adapted for selectively outputting a drive signal to the first laser stimulating ray source 21, the second laser stimulating ray source 22 or the third laser stimulating ray source 23 and outputting a drive signal to the filter unit motor 72.

The thus constituted scanner reads radiation data recorded in a stimulable phosphor sheet 10 by exposing the stimulable phosphor layer 12 to a radioactive labeling substance contained in a number of the absorptive regions 4 formed in the biochemical analysis unit 1 and produces biochemical analysis data in the following manner.

A stimulable phosphor sheet 10 is first set on the glass plate 41 of the stage 40 by a user.

An instruction signal indicating that radiation data recorded in the stimulable phosphor layer 12 formed on the support 11 of the stimulable phosphor sheet 10 are to be read is then input through the keyboard 71.

The instruction signal input through the keyboard 71 is input to the control unit 70 and the control unit 70 outputs a drive signal to the filter unit motor 72 in accordance with the instruction signal, thereby moving the filter unit 48 so as to locate the filter member 51d provided with the filter 52d having a property of transmitting only light having a wavelength corresponding to that of stimulated emission emitted from stimulable phosphor but cutting off light having a wavelength of 640 nm in the optical path of stimulated emission 45.

The control unit 70 then outputs a drive signal to the first laser stimulating ray source 21 to activate it, thereby causing it to emit a laser beam 24 having a wavelength of 640 nm.

The laser beam 24 emitted from the first laser stimulating ray source 21 is made a parallel beam by the collimator lens 25 and advances to the mirror 26 to be reflected thereby.

The laser beam 24 reflected by the mirror 26 passes through the first dichroic mirror 27 and the second dichroic mirror 28 and advances to the mirror 29.

The laser beam 24 advancing to the mirror 29 is reflected by the mirror 29 and further advances to a mirror 32 to be reflected thereby.

The laser beam 24 reflected by the mirror 32 passes through the hole 33 of the perforated mirror 34 and advances to the concave mirror 38.

The laser beam 24 advancing to the concave mirror 38 is reflected thereby and enters the optical head 35.

The laser beam 24 entering the optical head 35 is reflected by the mirror 36 and condensed by the aspherical lens 37 onto the stimulable phosphor layer 12 of the stimulable phosphor sheet 10 placed on the glass plate 41 of the stage 40.

As a result, a stimulable phosphor contained in the stimulable phosphor layer 12 formed on the support 11 of the stimulable phosphor sheet 10 is stimulated by the laser beam 24 and stimulated emission 45 is released from the stimulable phosphor.

The stimulated emission 45 released from the stimulable phosphor contained in the stimulable phosphor layer 12 is condensed by the aspherical lens 37 provided in the optical head 35 and reflected by the mirror 36 on the side of an optical path of the laser beam 24, thereby being made a parallel beam to advance to the concave mirror 38.

The stimulated emission 45 advancing to the concave mirror 38 is reflected by the concave mirror 38 and advances to the perforated mirror 34.

As shown in FIG. 7, the stimulated emission 45 advancing to the perforated mirror 34 is reflected downward by the perforated mirror 34 formed as a concave mirror and advances to the filter 52d of the filter unit 48.

Since the filter 52d has a property of transmitting only light having a wavelength corresponding to that of stimulated emission emitted from stimulable phosphor but cutting off light having a wavelength of 640 nm, light having a wavelength of 640 nm corresponding to that of the stimulating ray is cut off by the filter 52d and only light having a wavelength corresponding to that of stimulated emission passes through the filter 52d to be photoelectrically detected by the photommultiplier 50.

As described above, since the optical head 35 is moved on the base plate 63 in the X direction in FIG. 12 by the main scanning pulse motor 65 mounted on the base plate 63 and the base plate 63 is moved in the Y direction in FIG. 12 by the sub-scanning pulse motor 61, the whole surface of the stimulable phosphor layer 12 formed on the support 11 of the stimulable phosphor sheet 10 is scanned by the laser beam 24. Therefore, the photomultiplier 50 can read radiation data of a radioactive labeling substance recorded in the stimulable phosphor layer 12 by photoelectrically detecting the stimulated emission 45 released from stimulable phosphor contained in the stimulable phosphor layer 12 and produce analog data for biochemical analysis.

The analog data produced by photoelectrically detecting the stimulated emission 45 with the photomultiplier 50 are converted by the A/D converter 53 into digital data and the digital data are fed to the data processing apparatus 54.

On the other hand, when fluorescence data such as electrophoresis data of denatured DNA fragments labeled with a fluorescent substance such as a fluorescent dye recorded in a gel support or a transfer support are to be read to produce biochemical analysis data, a gel support or a transfer support is first set on the glass plate 41 of the stage 40 by a user.

A fluorescent substance identification signal for identifying the kind of fluorescent substance that is the labeling substance is then input through the keyboard 71 by the user together with an instruction signal indicating that fluorescence data are to be read.

When the kind of fluorescent substance is input by the user through the keyboard 71, the control unit 70 selects a laser stimulating ray source for emitting a laser beam 24 of a wavelength capable of efficiently stimulating the identified fluorescent substance from among the first laser stimulating ray source 21, the second laser stimulating ray source 22 and the third laser stimulating ray source 23 and selects the filter member for cutting light having a wavelength of the laser beam 24 to be used for stimulating the input fluorescent substance and transmitting light having a longer wavelength than that of the laser beam to be used for stimulation from among the three filter members 51a, 51b and 51c.

The whole surface of the gel support or the transfer support is then scanned with the laser beam 24 and fluorescence emission is photoelectrically detected by the photomultiplier 50 to produce analog data. The analog data are digitized by the A/D converter, thereby producing biochemical analysis data.

In this embodiment, the biochemical analysis unit 1 includes the substrate 2 formed of a metal such as stainless steel capable of attenuating radiation energy and light energy and having flexibility and formed with a number of the substantially circular through-holes 3, and a number of the absorptive regions 4 are formed by charging absorptive material such as nylon-6 in the through hole so that the difference between the surface of each absorptive region 4 and that of the substrate in the vertical direction, namely, the distance between the surface of each absorptive region 4 and that of the substrate is five times the diameter of the through-hole 3.

When biochemical analysis is performed, specific binding substances such as a plurality of cDNAs are spotted using a spotting device 5 in a number of absorption regions 4 of the biochemical analysis unit 1 and specific binding substances contained in a number of the absorptive regions 4 of the biochemical analysis unit 1 are selectively hybridized with a substance derived from a living organism labeled with a radioactive labeling substance. The stimulable phosphor sheet 10 is then superposed on the biochemical analysis unit 1 in such a manner that the stimulable phosphor layer 12 formed on the support 11 of the stimulable phosphor sheet 10 abuts against the surface of the substrate 2 of the biochemical analysis unit 1.

Therefore, according to this embodiment, since the substrate 2 of the biochemical analysis unit 1 is formed of a metal such as stainless steel capable of attenuating radiation energy, it is possible to effectively prevent electron beams (β rays) released from the radioactive labeling substance contained in the absorptive regions 4 in a number of through-holes 3 during the exposure operation from scattering in the substrate 2 of the biochemical analysis unit 1.

Further, according to this embodiment, since the absorptive region 4 is formed by charging absorptive material 4 in a number of the through-hole 3 so that the difference between the surface of each absorptive region 4 and that of the substrate 2 in the vertical direction, namely, the distance between the surface of each absorptive region 4 and that of the substrate 2 is five times the diameter of the through-hole 3, the electron beams (β rays) released from the radioactive labeling substance contained in the absorptive regions 4 in a number of the through-holes 3 are prevented by the collimation effect from broadening.

Therefore, since it is possible to selectively expose only the region of the stimulable phosphor layer 12 each of the absorptive regions 4 faces to the electron beams (β rays) released from the radioactive labeling substance contained in each of the absorptive regions 4, it is possible to prevent noise from being generated in biochemical analysis data produced by photoelectrically detecting stimulated emission released from the stimulable phosphor layer 12 in response to the stimulation with the laser beam 24 and to produce biochemical analysis data having a high quantitative accuracy.

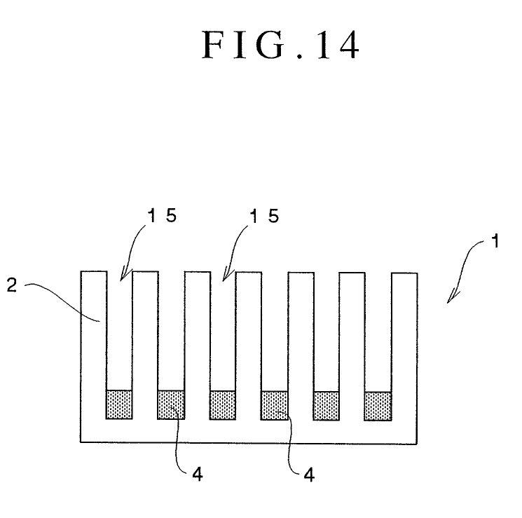

FIG. 14 is a schematic cross sectional view showing a biochemical analysis unit which is another preferred embodiment of the present invention.

As shown in FIG. 14, a biochemical analysis unit 1 according to this embodiment includes a substrate 2 formed of metal such as stainless steel capable of attenuating radiation energy and light energy and having flexibility and formed with a number of substantially circular recesses 15 at a high density, and a number of absorptive regions 4 are dot-like formed by charging absorptive material such as nylon-6 in the recesses 15.

Although not shown in FIG. 14, in this embodiment, the recesses 15 are formed in the substrate 2 so that substantially circular absorptive regions having a size of about 0.07 cm² are regularly formed in the manner of a matrix of 120 columns×160 lines and, therefore, 19,200 absorptive regions 4 are formed.

As shown in FIG. 14, in this embodiment, the absorptive region 4 is formed by charging absorptive material 4 in a number of the recesses 15 so that the difference between the surface of each absorptive region 4 and that of the substrate in the vertical direction, namely, the distance between the surface of each absorptive region 4 and that of the substrate is three times the diameter of the recess 15.

Similarly to the biochemical analysis unit 1 shown in FIGS. 1 and 2, in this embodiment, specific binding substances such as a plurality of cDNAs are spotted using a spotting device 5 shown in FIG. 3 in the absorption regions 4 formed in a number of the recesses 15 and as shown in FIG. 4, the biochemical analysis unit 1 is bent and accommodated in the hybridization reaction vessel 8 containing a substance derived from a living organism labeled with a radioactive labeling substance along the inner wall surface thereof, thereby selectively hybridizing a substance derived from a living organism labeled with a radioactive labeling substance and contained in the hybridization reaction vessel 8 with specific binding substances spotted in the absorption regions 4 formed in a number of the recesses 15.

As shown in FIG. 5, the stimulable phosphor sheet 10 is superposed on the biochemical analysis unit 1 in such a manner that the stimulable phosphor layer 12 uniformly formed on one surface of the support 11 of the stimulable phosphor sheet 10 abuts against the surface of the substrate 2 of the biochemical analysis unit 1, thereby exposing the stimulable phosphor layer 12 formed on the support 11 of the stimulable phosphor sheet 10 to a radioactive labeling substance contained in the absorptive regions 4 in a number of the recesses 15.

Therefore, according to this embodiment, since the substrate 2 of the biochemical analysis unit 1 is formed of a metal such as stainless steel capable of attenuating radiation energy, it is possible to effectively prevent electron beams (β rays) released from the radioactive labeling substance contained in the absorptive regions 4 in a number of recesses 15 during the exposure operation from scattering in the substrate 2 of the biochemical analysis unit 1.

Further, according to this embodiment, since the absorptive region 4 is formed by charging absorptive material 4 in a number of the recesses 15 so that the difference between the surface of each absorptive region 4 and that of the substrate 2 in the vertical direction, namely, the distance between the surface of each absorptive region 4 and that of the substrate 2 is three times the diameter of the recess 15, the electron beams (β rays) released from the radioactive labeling substance contained in the absorptive regions 4 in a number of the recesses 15 are prevented by the collimation effect from broadening.

Therefore, since it is possible to selectively expose only the region of the stimulable phosphor layer 12 each of the absorptive regions 4 faces to the electron beams (β rays) released from the radioactive labeling substance contained in each of the absorptive regions 4, it is possible to prevent noise from being generated in biochemical analysis data produced by photoelectrically detecting stimulated emission released from the stimulable phosphor layer 12 in response to the stimulation with the laser beam 24 and to produce biochemical analysis data having a high quantitative accuracy.

The present invention has thus been shown and described with reference to specific embodiments. However, it should be noted that the present invention is in no way limited to the details of the described arrangements but changes and modifications may be made without departing from the scope of the appended claims.

For example, in the above-described embodiments, as specific binding substances, cDNAs each of which has a known base sequence and is different from the others are used. However, specific binding substances usable in the present invention are not limited to cDNAs but all specific binding substances capable of specifically binding with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, can be employed in the present invention as a specific binding substance.

Further, in the above-described embodiments, although specific binding substances are hybridized with substances derived from a living organism labeled with a radioactive labeling substance, it is not absolutely necessary to hybridize substances derived from a living organism with specific binding substances and substances derived from a living organism may be specifically bound with specific binding substances by means of antigen-antibody reaction, receptor-ligand reaction or the like instead of hybridization.

Furthermore, in the above-described embodiments, although the substrate 2 has flexibility, it is not absolutely necessary to form the substrate 2 so as to be flexible.

Further, in the above described embodiments, although 19,200 of substantially circular absorptive regions 4 having a size of about 0.07 cm² are regularly formed in the biochemical analysis unit 1 in the manner of a matrix of 120 columns×160 lines, the number or size of the absorptive regions 4 may be arbitrarily selected in accordance with the purpose. Preferably, 10 or more of the absorptive regions 4 having a size of 5 cm² or less are formed in the biochemical analysis unit 1 at a density of 10/ cm² or less.

Furthermore, in the above described embodiments, although 19,200 of substantially circular absorptive regions 4 having a size of about 0.07 cm² are regularly formed in the biochemical analysis unit 1 in the manner of a matrix of 120 columns×160 lines, it is not absolutely necessary to regularly form the absorptive regions 4 in the biochemical analysis unit 1.

Moreover, in the above described embodiments, although each of the absorptive regions 4 are formed substantially circular, the shape of each of the absorptive regions 4 is not limited to substantially a circular shape and may be arbitrarily selected.

Further, in the embodiment shown in FIGS. 1 to 13, the absorptive region 4 is formed by charging absorptive material 4 in a number of the through-holes 3 so that the difference between the surface of each absorptive region 4 and that of the substrate 2 in the vertical direction, namely, the distance between the surface of each absorptive region 4 and that of the substrate 2 is five times the diameter of the through-hole 3 and in the embodiment shown in FIG. 14, the absorptive region 4 is formed by charging absorptive material 4 in a number of the recesses 15 so that the difference between the surface of each absorptive region 4 and that of the substrate 2 in the vertical direction, namely, the distance between the surface of each absorptive region 4 and that of the substrate 2 is three times the diameter of the recess 15. However, it is not absolutely necessary to form the absorptive region 4 by charging absorptive material 4 in the through-hole 3 so that the difference between the surface of each absorptive region 4 and that of the substrate 2 in the vertical direction, namely, the distance between the surface of each absorptive region 4 and that of the substrate 2 is five times the diameter of the through-hole 3 and it is not also absolutely necessary to form the absorptive region 4 by charging absorptive material 4 in the recesses 15 so that the difference between the surface of each absorptive region 4 and that of the substrate 2 in the vertical direction, namely, the distance between the surface of each absorptive region 4 and that of the substrate 2 is three times the diameter of the recess 15. The difference between the surface of each absorptive region 4 and that of the substrate 2 in the vertical direction, namely, the distance between the surface of each absorptive region 4 and that of the substrate 2 may be 0.5 to 100 times, preferably 1 to 10 times the diameter of the through-hole 3 or the recess 15 and the difference between the surface of each absorptive region 4 and that of the substrate 2 in the vertical direction, namely, the distance between the surface of each absorptive region 4 and that of the substrate 2 can be arbitrarily selected in this range.

Furthermore, in the above described embodiments, the stimulable phosphor sheet 10 including the stimulable phosphor layer 12 uniformly formed on one surface of the support 11 is superposed on the biochemical analysis unit 1, thereby exposing the stimulable phosphor layer 12 to a radioactive labeling substance contained in the absorptive regions 4 of the biochemical analysis unit 1. However, it is possible to superpose a stimulable phosphor sheet 10 including a number of stimulable phosphor layer regions formed by charging stimulable phosphor in a number of holes formed in the support 11 in the same pattern as that of a number of the through-holes or the recesses 15 formed in the biochemical analysis unit 1 on the biochemical analysis unit 1 in such a manner that the surfaces of a number of the stimulable phosphor layer regions face the corresponding absorptive regions 4 formed in the substrate 2 of the biochemical analysis unit 1, thereby exposing a number of the stimulable phosphor layer regions to a radioactive labeling substance contained in the absorptive regions 4 of the biochemical analysis unit 1.

Further, in the above described embodiments, the hybridization reaction solution 9 containing a substance derived from a living organism and labeled with a radioactive labeling substance is prepared and the substance derived from a living organism and labeled with the radioactive labeling substance is hybridized with specific binding substances contained in a number of the absorptive regions 4 of the biochemical analysis unit 1, whereby radiation data are recorded in a number of the absorptive regions 4 of the biochemical analysis unit 1. The radiation data recorded in a number of the absorptive regions 4 of the biochemical analysis unit 1 are transferred onto the stimulable phosphor layer 12 formed on the support 11 of the stimulable phosphor sheet 10 and the radiation data transferred onto the stimulable phosphor layer 12 of the stimulable phosphor sheet 10 are read by the scanner shown in FIGS. 6 to 13, thereby oroducing biochemical analysis data. However, it is also possible to produce biochemical analysis data by preparing a hybridization reaction solution 9 containing a substance derived from a living organism and labeled with a labeling substance which generates chemiluminescent emission when it contacts a chemiluminescent substrate, hybridizing the substance derived from a living organism and labeled with the labeling substance which generates chemiluminescent emission when it contacts a chemiluminescent substrate with specific binding substances contained in a number of the absorptive regions 4 of the biochemical analysis unit 1, thereby recording chemiluminescent data in a number of the absorptive regions 4 of the biochemical analysis unit 1, causing a chemiluminescent substrate to come into contact with a number of the absorptive regions 4 of the biochemical analysis unit 1, thereby causing a number of the absorptive regions 4 of the biochemical analysis unit 1 to release chemiluminescent emission, superposing the stimulable phosphor layer 12 formed on the support 11 of the stimulable phosphor sheet 10 on the biochemical analysis unit 1 whose absorptive regions are releasing chemiluminescent emission, exposing the stimulable phosphor layer 12 of the stimulable phosphor sheet 10 to chemiluminescent emission released from a number of the absorptive regions 4 of the biochemical analysis unit 1, thereby storng the energy of chemiluminescent emission in the stimulable phosphor layer 12 of the stimulable phosphor sheet 10, scanning the stimulable phosphor layer 12 of the stimulable phosphor sheet 10 with the laser beam 24 using the scanner shown in FIGS. 6 to 13, photoelectrically detecting stimulated emission 45 released from the stimulable phosphor layer 12 of the stimulable phosphor sheet 10 to read the chemiluminescent data. In the case where the stimulable phosphor layer 12 of the stimulable phosphor sheet 10 is exposed to chemiluminescent emission released from a number of the absorptive regions 4 of the biochemical analysis unit 1, since a number of the absorptive regions 4 of the biochemical analysis unit 1 are formed by charging nylon-6 in a number of the through-holes 3 formed in the substrate 2 made of stainless steel capable of attenuating light energy, it is also possible to effectively prevent chemiluminescent emission released from a number of the absorptive regions 4 of the biochemical analysis unit 1 from being scattered in the substrate 2 of the biochemical analysis unit 1 and to effectively prevent chemiluminescent emission released from the absorptive regions 4 in a number of the through-hole 3 by the collimation effect from broadening. Therefore, it is possible to effectively prevent noise from being generated in biochemical analysis data produced by photoelectrically detecting stimulated emission released from the stimulable phosphor layer in response to the stimulation with a stimulating ray and to produce biochemical analysis data having a high quantitative accuracy.

Moreover, in the above described embodiments, although the substrate 2 of the biochemical analysis unit 1 is made of a metal such as stainless steel, it is sufficient for the substrate 2 to be made of a material capable of attenuating radiation energy and the substrate 2 can be formed of either inorganic compound material or organic compound material and is preferably formed of metal material, ceramic material or plastic material. Illustrative examples of inorganic compound materials include metals such as gold, silver, copper, zinc, aluminum, titanium, tantalum, chromium, steel, nickel, cobalt, lead, tin, selenium and the like; alloys such as brass, stainless, bronze and the like; silicon materials such as silicon, amorphous silicon, glass, quartz, silicon carbide, silicon nitride and the like; metal oxides such as aluminum oxide, magnesium oxide, zirconium oxide and the like; and inorganic salts such as tungsten carbide, calcium carbide, calcium sulfate, hydroxy apatite, gallium arsenide and the like. High molecular compounds are preferably used as organic compound material and illustrative examples thereof include polyolefins such as polyethylene, polypropylene and the like; acrylic resins such as polymethyl methacrylate, polybutylacrylate/polymethyl methacrylate copolymer and the like; polyacrylonitrile; polyvinyl chloride; polyvinylidene chloride; polyvinylidene fluoride; polytetrafluoroethylene; polychlorotrifluoroethylene; polycarbonate; polyesters such as polyethylene naphthalate, polyethylene terephthalate and the like; nylons such as nylon-6, nylon-6,6, nylon-4, 10 and the like; polyimide; polysulfone; polyphenylene sulfide; silicon resins such as polydiphenyl siloxane and the like; phenol resins such as novolac and the like; epoxy resin; polyurethane; polystyrene, butadiene-styrene copolymer; polysaccharides such as cellulose, acetyl cellulose, nitrocellulose, starch, calcium alginate, hydroxypropyl methyl cellulose and the like; chitin; chitosan; urushi (Japanese lacquer); polyamides such as gelatin, collagen, keratin and the like; and copolymers of these high molecular materials.

Further, in the above described embodiments, although the absorptive regions 4 of the biochemical analysis unit 1 are formed of nylon-6, the absorptive material for forming the absorptive regions 4 of the biochemical analysis unit 1 is not limited to nylon-6 and other kinds of absorptive materials can be employed instead for forming the absorptive regions 4 of the biochemical analysis unit 1. A porous material or a fiber material may be preferably used as the absorptive material for forming the absorptive regions 4 of the biochemical analysis unit 1. Otherwise the absorptive regions 4 of the biochemical analysis unit 1 may be formed by combining a porous material and a fiber material. A porous material used for forming the absorptive regions 4 of the biochemical analysis unit 1 may be any type of organic material or inorganic material and may be an organic/inorganic composite material. An organic porous material used for forming the absorptive regions 4 of the biochemical analysis unit 1 is not particularly limited but a carbon porous material such as an activated carbon or a porous material capable of forming a membrane filter is preferably used. Illustrative examples of porous materials capable of forming a membrane filter include nylons such as nylon-6, nylon-6,6, nylon-4,10; cellulose derivatives such as nitrocellulose, acetyl cellulose, butyric-acetyl cellulose; collagen; alginic acids such as alginic acid, calcium alginate, alginic acid/poly-L-lysine polyionic complex; polyolefins such as polyethylene, polypropylene; polyvinyl chloride; polyvinylidene chloride; polyfluorides such as polyvinylidene fluoride, polytetrafluoride; and copolymers or composite materials thereof. An inorganic porous material used for forming the absorptive regions 4 of the biochemical analysis unit 1 is not particularly limited. Illustrative examples of inorganic porous materials preferably usable in the present invention include metals such as platinum, gold, iron, silver, nickel, aluminum and the like; metal oxides such as alumina, silica, titania, zeolite and the like; metal salts such as hydroxy apatite, calcium sulfate and the like; and composite materials thereof. A fiber material used for forming the absorptive regions 4 of the biochemical analysis unit 1 is not particularly limited. Illustrative examples of fiber materials preferably usable in the present invention include nylons such as nylon-6, nylon-6,6, nylon-4,10; and cellulose derivatives such as nitrocellulose, acetyl cellulose, butyric-acetyl cellulose.

According to the present invention, it is possible to provide a biochemical analysis unit and a method for exposing a stimulable phosphor sheet using the same which can prevent noise caused by the scattering of electron beams released from a radioactive labeling substance from being generated in biochemical analysis data even in the case of forming a plurality of spot-like regions containing specific binding substances on the surface of a carrier at a high density, which can specifically bind with a substance derived from a living organism and whose sequence, base length, composition and the like are known, specifically binding the specific binding substances contained in the plurality of spot-like regions with a substance derived from a living organism labeled with a radioactive substance to selectively label the spot-like specific binding substances with the radioactive substance, thereby obtaining a biochemical analysis unit, superposing the thus obtained biochemical analysis unit and a stimulable phosphor layer, exposing the stimulable phosphor layer to the radioactive labeling substance, irradiating the stimulable phosphor layer with a stimulating ray to excite the stimulable phosphor, photoelectrically detecting the stimulated emission released from the stimulable phosphor layer to produce biochemical analysis data, and analyzing the substance derived from a living organism.

Further, according to the present invention, it is possible to provide a biochemical analysis unit and a method for exposing a stimulable phosphor sheet using the same which can prevent noise caused by the scattering of chemiluminescent emission released from a plurality of spot-like regions of a biochemical analysis unit from being generated in biochemical analysis data even in the case of forming the plurality of spot-like regions containing specific binding substances on the surface of a carrier at a high density, which can specifically bind with a substance derived from a living organism and whose sequence, base length, composition and the like are known, specifically binding the specific binding substances contained in the plurality of spot-like regions with a substance derived from a living organism labeled with a labeling substance which generates chemiluminescent emission when it contacts a chemiluminescent substrate to produce a biochemical analysis unit, causing a chemiluminescent substrate to come into contact with the biochemical analysis unit, thereby causing the plurality of spot-like regions of the biochemical analysis unit to release chemiluminescent emission, holding the biochemical analysis unit whose plurality of spot-like regions are releasing chemiluminescent emission in close contact with a stimulable phosphor layer, exposing the stimulable phosphor layer to chemiluminescent emission, irradiating the stimulable phosphor layer with a stimulating ray, photoelectrically detecting stimulated emission released from the stimulable phosphor layer to produce biochemical analysis data, and analyzing the substance derived from a living organism.

1. A biochemical analysis unit comprising a plurality of absorptive regions formed of absorptive material and spaced apart from each other and a plurality of isolating regions formed of a material capable of attenuating radiation energy and/or light energy for isolating the plurality of absorptive regions, the plurality of isolating regions being formed so that surfaces thereof lie outward of surfaces of the individual absorptive regions.

2. A biochemical analysis unit in accordance with claim 1 wherein the plurality of absorptive regions of the biochemical analysis unit are formed by charging absorptive material in a plurality of holes formed spaced apart from each other in a substrate made of a material capable of attenuating radiation energy and/or light energy and the plurality of isolating regions are constituted by the substrate.

3. A biochemical analysis unit in accordance with claim 2 wherein the plurality of absorptive regions of the biochemical analysis unit are formed by charging absorptive material in a plurality of through-holes formed spaced apart from each other in a substrate made of a material capable of attenuating radiation energy and/or light energy and the plurality of isolating regions are constituted by the substrate.

4. A biochemical analysis unit in accordance with claim 2 wherein the plurality of absorptive regions of the biochemical analysis unit are formed by charging absorptive material in a plurality of recesses formed spaced apart from each other in a substrate made of a material capable of attenuating radiation energy and/or light energy and the plurality of isolating regions are constituted by the substrate.

5. A biochemical analysis unit in accordance with claim 1 wherein the plurality of isolating regions of the biochemical analysis unit are formed in such a manner that the surfaces thereof lie outward of the surfaces of the respective absorptive regions by 0.5 to 100 times the maximum width of each of the absorptive regions.

6. A biochemical analysis unit in accordance with claim 2 wherein the plurality of isolating regions of the biochemical analysis unit are formed in such a manner that the surfaces thereof lie outward of the surfaces of the respective absorptive regions by 0.5 to 100 times the maximum width of each of the absorptive regions.

7. A biochemical analysis unit in accordance with claim 5 wherein the plurality of isolating regions of the biochemical analysis unit are formed in such a manner that the surfaces thereof lie outward of the surfaces of the respective absorptive regions by 1 to 10 times the maximum width of each of the absorptive regions.

8. A biochemical analysis unit in accordance with claim 6 wherein the plurality of isolating regions of the biochemical analysis unit are formed in such a manner that the surfaces thereof lie outward of the surfaces of the respective absorptive regions by 1 to 10 times the maximum width of each of the absorptive regions.

9. A biochemical analysis unit in accordance with claim 1 wherein the biochemical analysis unit is formed with 10 or more absorptive regions.

10. A biochemical analysis unit in accordance with claim 2 wherein the biochemical analysis unit is formed with 10 or more absorptive regions.

11. A biochemical analysis unit in accordance with claim 9 wherein the biochemical analysis unit is formed with 1000 or more absorptive regions.

12. A biochemical analysis unit in accordance with claim 10 wherein the biochemical analysis unit is formed with 1000 or more absorptive regions.

13. A biochemical analysis unit in accordance with claim 1 wherein each of the plurality of absorptive regions formed in the biochemical 5 analysis unit has a size of less than 5 mm².

14. A biochemical analysis unit in accordance with claim 2 wherein each of the plurality of absorptive regions formed in the biochemical analysis unit has a size of less than 5 mm².

15. A biochemical analysis unit in accordance with claim 13 wherein each of the plurality of absorptive regions formed in the biochemical analysis unit has a size of less than 1 mm².

16. A biochemical analysis unit in accordance with claim 14 wherein each of the plurality of absorptive regions formed in the biochemical analysis unit has a size of less than 1 mm².

17. A biochemical analysis unit in accordance with claim 1 wherein the plurality of absorptive regions are formed in the biochemical analysis unit at a density of 10 or more per cm².

18. A biochemical analysis unit in accordance with claim 2 wherein the plurality of absorptive regions are formed in the biochemical analysis unit at a density of 10 or more per cm².

19. A biochemical analysis unit in accordance with claim 17 wherein the plurality of absorptive regions are formed in the biochemical analysis unit at a density of 1,000 or more per cm².

20. A biochemical analysis unit in accordance with claim 18 wherein the plurality of absorptive regions are formed in the biochemical analysis unit at a density of 1,000 or more per cm².

21. A biochemical analysis unit in accordance with claim 1 wherein the isolating region of the biochemical analysis unit has a property of reducing the energy of radiation and/or the energy of light to ⅕ or less when the radiation and/or light travels in the isolating region by a distance equal to that between neighboring absorptive regions.

22. A biochemical analysis unit in accordance with claim 2 wherein the isolating region of the biochemical analysis unit has a property of reducing the energy of radiation and/or the energy of light to ⅕ or less when the radiation and/or light travels in the isolating region by a distance equal to that between neighboring absorptive regions.

23. A biochemical analysis unit in accordance with claim 21 wherein the isolating region of the biochemical analysis unit has a property of reducing the energy of radiation and/or the energy of light to 1/100 or less when the radiation and/or light travels in the isolating region by a distance equal to that between neighboring absorptive regions.

24. A biochemical analysis unit in accordance with claim 22 wherein the isolating region of the biochemical analysis unit has a property of reducing the energy of radiation and/or the energy of light to 1/100 or less when the radiation and/or light travels in the isolating region by a distance equal to that between neighboring absorptive regions.

25. A biochemical analysis unit in accordance with claim 1 wherein the isolating region of the biochemical analysis unit is formed of a material selected from a group consisting of a metal material, a ceramic material or a plastic material.

26. A biochemical analysis unit in accordance with claim 2 wherein the isolating region of the biochemical analysis unit is formed of a material selected from a group consisting of a metal material, a ceramic material or a plastic material.

27. A biochemical analysis unit in accordance with claim 1 wherein the isolating region of the biochemical analysis unit is formed of a metal material.

28. A biochemical analysis unit in accordance with claim 2 wherein the isolating region of the biochemical analysis unit is formed of a metal material.

29. A biochemical analysis unit in accordance with claim 1 wherein the absorptive regions of the biochemical analysis unit is formed of a porous material or a fiber material.

30. A biochemical analysis unit in accordance with claim 2 wherein the absorptive regions of the biochemical analysis unit is formed of a porous material or a fiber material.

31. A method for exposing a stimulable phosphor sheet comprising the step of superposing a biochemical analysis unit including a plurality of absorptive regions formed of absorptive material and spaced apart from each other and a plurality of isolating regions formed of a material capable of attenuating radiation energy for isolating the plurality of absorptive regions, the plurality of isolating regions being formed so that surfaces thereof lie outward of surfaces of individual absorptive regions, and prepared by spotting specific binding substances whose sequence, base length, composition and the like are known in the plurality of absorptive regions and specifically binding a substance derived from a living organism and labeled with a radioactive substance with the specific binding substances, thereby selectively labeling the plurality of absorptive regions, on a stimulable phosphor layer formed on a stimulable phosphor sheet in such a manner that the plurality of isolating regions are in contact with the stimulable phosphor layer formed on the stimulable phosphor sheet, thereby exposing the stimulable phosphor layer of the stimulable phosphor sheet to the radioactive labeling substance selectively contained in the plurality of absorptive regions of the biochemical analysis unit.

32. A method for exposing a stimulable phosphor sheet in accordance with claim 31 wherein the plurality of absorptive regions of the biochemical analysis unit are formed by charging absorptive material in a plurality of holes formed spaced apart from each other in a substrate made of a material capable of attenuating radiation energy and the plurality of isolating regions are constituted by the substrate.

33. A method for exposing a stimulable phosphor sheet in accordance with claim 31 wherein the plurality of isolating regions of the biochemical analysis unit are formed in such a manner that the surfaces thereof lie outward of the surfaces of the respective absorptive regions by 0.5 to 100 times the maximum width of each of the absorptive regions.

34. A method for exposing a stimulable phosphor sheet in accordance with claim 32 wherein the plurality of isolating regions of the biochemical analysis unit are formed in such a manner that the surfaces thereof lie outward of the surfaces of the respective absorptive regions by 0.5 to 100 times the maximum width of each of the absorptive regions.

35. A method for exposing a stimulable phosphor sheet in accordance with claim 31 wherein the plurality of isolating regions of the biochemical analysis unit are formed in such a manner that the surfaces thereof lie outward of the surfaces of the respective absorptive regions by 1 to 10 times the maximum width of each of the absorptive regions.

36. A method for exposing a stimulable phosphor sheet in accordance with claim 32 wherein the plurality of isolating regions of the biochemical analysis unit are formed in such a manner that the surfaces thereof lie outward of the surfaces of the respective absorptive regions by 1 to 10 times the maximum width of each of the absorptive regions.

37. A method for exposing a stimulable phosphor sheet in accordance with claim 31 wherein the biochemical analysis unit is formed with 10 or more absorptive regions.

38. A method for exposing a stimulable phosphor sheet in accordance with claim 32 wherein the biochemical analysis unit is formed with 10 or more absorptive regions.

39. A method for exposing a stimulable phosphor sheet in accordance with claim 31 wherein each of the plurality of absorptive regions formed in the biochemical analysis unit has a size of less than 5 mm².

40. A method for exposing a stimulable phosphor sheet in accordance with claim 32 wherein each of the plurality of absorptive regions formed in the biochemical analysis unit has a size of less than 5 mm².

41. A method for exposing a stimulable phosphor sheet in accordance with claim 31 wherein the plurality of absorptive regions are formed in the biochemical analysis unit at a density of 10 or more per cm².

42. A method for exposing a stimulable phosphor sheet in accordance with claim 32 wherein the plurality of absorptive regions are formed in the biochemical analysis unit at a density of 10 or more per cm².

43. A method for exposing a stimulable phosphor sheet in accordance with claim 31 wherein the isolating region of the biochemical analysis unit has a property of reducing the energy of radiation to ⅕ or less when the radiation travels in the isolating region by a distance equal to that between neighboring absorptive regions.

44. A method for exposing a stimulable phosphor sheet in accordance with claim 32 wherein the isolating region of the biochemical analysis unit has a property of reducing the energy of radiation to ⅕ or less when the radiation travels in the isolating region by a distance equal to that between neighboring absorptive regions.

45. A method for exposing a stimulable phosphor sheet in accordance with claim 31 wherein the isolating region of the biochemical analysis unit is formed of a material selected from a group consisting of a metal material, a ceramic material or a plastic material.

46. A method for exposing a stimulable phosphor sheet in accordance with claim 32 wherein the isolating region of the biochemical analysis unit is formed of a material selected from a group consisting of a metal material, a ceramic material or a plastic material.

47. A method for exposing a stimulable phosphor sheet in accordance with claim 31 wherein the isolating region of the biochemical analysis unit is formed of a metal material.

48. A method for exposing a stimulable phosphor sheet in accordance with claim 32 wherein the isolating region of the biochemical analysis unit is formed of a metal material.

49. A method for exposing a stimulable phosphor sheet in accordance with claim 31 wherein the absorptive regions of the biochemical analysis unit is formed of a porous material or a fiber material.

50. A method for exposing a stimulable phosphor sheet in accordance with claim 32 wherein the absorptive regions of the biochemical analysis unit is formed of a porous material or a fiber material.

51. A method for exposing a stimulable phosphor sheet comprising the step of causing a biochemical analysis unit including a plurality of absorptive regions formed of absorptive material and spaced apart from each other and a plurality of isolating regions formed of a material capable of attenuating light energy for isolating the plurality of absorptive regions, the plurality of isolating regions being formed so that surfaces thereof lie outward of surfaces of individual absorptive regions, and prepared by spotting specific binding substances whose sequence, base length, composition and the like are known in the plurality of absorptive regions and specifically binding a substance derived from a living organism and labeled with a labeling substance which generates chemiluminescent emission when it contacts a chemiluminescent substrate with the specific binding substances, thereby selectively labeling the plurality of absorptive regions, to come into contact with a chemiluminescent substrate, thereby causing the plurality of absorptive regions to release chemiluminescent emission, superposing the biochemical analysis unit whose plurality of absorptive regions are releasing chemiluminescent emission with a stimulable phosphor layer of a stimulable phosphor sheet in such a manner that the plurality of isolating regions abut against the surface of the stimulable phosphor layer formed on the stimulable phosphor sheet and exposing the stimulable phosphor layer of the stimulable phosphor sheet to chemiluminescent emission selectively released from the plurality of absorptive regions of the biochemical analysis unit.

52. A method for exposing a stimulable phosphor sheet in accordance with claim 51 wherein the plurality of absorptive regions of the biochemical analysis unit are formed by charging absorptive material in a plurality of holes formed spaced apart from each other in a substrate made of a material capable of attenuating light energy and the plurality of isolating regions are constituted by the substrate.

53. A method for exposing a stimulable phosphor sheet in accordance with claim 51 wherein the plurality of isolating regions of the biochemical analysis unit are formed in such a manner that the surfaces thereof lie outward of the surfaces of the respective absorptive regions by 0.5 to 100 times the maximum width of each of the absorptive regions.

54. A method for exposing a stimulable phosphor sheet in accordance with claim 52 wherein the plurality of isolating regions of the biochemical analysis unit are formed in such a manner that the surfaces thereof lie outward of the surfaces of the respective absorptive regions by 0.5 to 100 times the maximum width of each of the absorptive regions.

55. A method for exposing a stimulable phosphor sheet in accordance with claim 51 wherein the plurality of isolating regions of the biochemical analysis unit are formed in such a manner that the surfaces thereof lie outward of the surfaces of the respective absorptive regions by 1 to 10 times the maximum width of each of the absorptive regions.

56. A method for exposing a stimulable phosphor sheet in accordance with claim 52 wherein the plurality of isolating regions of the biochemical analysis unit are formed in such a manner that the surfaces thereof lie outward of the surfaces of the respective absorptive regions by 1 to 10 times the maximum width of each of the absorptive regions.

57. A method for exposing a stimulable phosphor sheet in accordance with claim 51 wherein the biochemical analysis unit is formed with 10 or more absorptive regions.

58. A method for exposing a stimulable phosphor sheet in accordance with claim 52 wherein the biochemical analysis unit is formed with 10 or more absorptive regions.

59. A method for exposing a stimulable phosphor sheet in accordance with claim 51 wherein each of the plurality of absorptive regions formed in the biochemical analysis unit has a size of less than 5 mm².

60. A method for exposing a stimulable phosphor sheet in accordance with claim 52 wherein each of the plurality of absorptive regions formed in the biochemical analysis unit has a size of less than 5 mm².

61. A method for exposing a stimulable phosphor sheet in accordance with claim 51 wherein the plurality of absorptive regions are formed in the biochemical analysis unit at a density of 10 or more per cm².

62. A method for exposing a stimulable phosphor sheet in accordance with claim 52 wherein the plurality of absorptive regions are formed in the biochemical analysis unit at a density of 10 or more per cm².

63. A method for exposing a stimulable phosphor sheet in accordance with claim 51 wherein the isolating region of the biochemical analysis unit has a property of reducing the energy of light to ⅕ or less when the light travels in the isolating region by a distance equal to that between neighboring absorptive regions.

64. A method for exposing a stimulable phosphor sheet in accordance with claim 52 wherein the isolating region of the biochemical analysis unit has a property of reducing the energy of light to ⅕ or less when the light travels in the isolating region by a distance equal to that between neighboring absorptive regions.

65. A method for exposing a stimulable phosphor sheet in accordance with claim 51 wherein the isolating region of the biochemical analysis unit is formed of a material selected from a group consisting of a metal material, a ceramic material or a plastic material.

66. A method for exposing a stimulable phosphor sheet in accordance with claim 52 wherein the isolating region of the biochemical analysis unit is formed of a material selected from a group consisting of a metal material, a ceramic material or a plastic material.

67. A method for exposing a stimulable phosphor sheet in accordance with claim 51 wherein the isolating region of the biochemical analysis unit is formed of a metal material.

68. A method for exposing a stimulable phosphor sheet in accordance with claim 52 wherein the isolating region of the biochemical analysis unit is formed of a metal material.

69. A method for exposing a stimulable phosphor sheet in accordance with claim 51 wherein the absorptive regions of the biochemical analysis unit is formed of a porous material or a fiber material.

70. A method for exposing a stimulable phosphor sheet in accordance with claim 52 wherein the absorptive regions of the biochemical analysis unit is formed of a porous material or a fiber material.
